(19) United States
(12) Patent Application Publication
(10) Pub. No.: US 2018/0071434 A1
(43) Pub. Date: Mar. 15, 2018

(54) SCAFFOLDS AND METHODS OF MAKING AND USING THE SAME

(71) Applicant: TRUSTEES OF TUFTS COLLEGE, Medford, MA (US)

(72) Inventors: Fiorenzo G. Omenetto, Lexington, MA (US); David L. Kaplan, Concord, MA (US); Matthew B. Applegate, Somerville, MA (US)

(21) Appl. No.: 15/266,590

(22) Filed: Sep. 15, 2016

(51) Int. Cl. A61L 27/52; A61L 27/22; A61L 27/38

(52) U.S. Cl. CPC A61L 27/52; A61L 2300/64; A61L 27/38; A61L 27/22

(57) ABSTRACT

The present disclosure relates to scaffolds such as protein hydrogel scaffolds. The present disclosure provides methods and technologies that permit formation of cavities within hydrogel scaffolds; in some embodiments, such technologies permit controlled formation of cavities of particular predetermined shape and/or arrangement. In particular embodiments, the present disclosure provides multiphoton absorption technologies that achieve production of cavity-containing scaffolds.

### GOVERNMENT SUPPORT

This invention was made with government support under grant No. N00014-13-1-0596, awarded by the Office of Naval Research. The government has certain rights in the invention.

### BACKGROUND

Hydrogels have a variety of important applications, including as scaffolds for cell growth, and/or cell or tissue engineering, controlled degradation applications, controlled release applications, including drug delivery, drug release, preservation of biologically labile compounds, sensors, tissue regeneration, tissue scaffolding, triggered release applications. Hydrogels are processed with different materials and methods such that they may be tuned to possess a broad range of mechanical properties, such as elasticity, resilience, stiffness, and swelling.

### SUMMARY

Among other things, the present disclosure provides methods and technologies that permit production of scaffolds, such as biomechanical scaffolds. Provided scaffolds are characterized by unique features that provide advantages over existing structures. In particular, the present disclosure provides methods and technologies that form scaffolds that are characterized by a presence of one or more defined cavities present beneath a surface of a material.

The present disclosure encompasses an insight that technologies that introduce pores and/or networks of pores are useful but that these technologies suffer from randomness due to the inability to design or structure such networks in having a predetermined location, shape, size, structure, or arrangement. Technologies and methods provided by the present disclosure provide can do this.

In some embodiments, provided methods and technologies permit production of scaffolds with uniquely desirable and useful structural and functional characteristics. In some embodiments, these cavities can be formed at a specific depth and/or location. In some embodiments, such cavities can be formed having a precisely predetermined shape, size, structure, and/or arrangement relative to one another.

These methods and technologies are particularly useful, for example, in the context of tissue engineering applications or sensor applications. Depending on their application, these characteristic cavities are or include channels, conduits, microvasculature, openings, tunnels, vasculature, or voids.

In some embodiments, provided methods and technologies include removing a portion of material to form a cavity. In some embodiments, provided methods and technologies of forming cavities include shaping materials. In some embodiments, for example, provided methods and technologies include removing material or shaping a material to form scaffolds for sensors, tissue engineering, or tissue guided regeneration applications.

In some embodiments, provided scaffolds are particularly suitable as soft biomaterials that characterized by physical and mechanical properties that are compatible to match a broad range of human tissues. Implementations of the present disclosure are useful for a wide range of applications, including but not limited to: biomedical applications, regenerative medicine, tissue regeneration, and tissue engineering applications.

Indeed, in some embodiments, provided methods and technologies are capable of forming scaffolds that are cytocompatible and capable of supporting and sustaining cells and/or cellular activity.

In some embodiments, methods and technologies as provided herein produce scaffolds that are biocompatible such that they are able to stabilize biologically active molecules and macromolecules and/or biologically labile compounds, such as enzymes.

In some embodiments, methods and technologies as provided herein produce scaffolds that are biodegradable. In some embodiments, methods and technologies as provided herein produce scaffolds that are programmably biodegradable such that these scaffolds will degrade after a specific length of time or in response to a trigger.

In some embodiments, provided methods and technologies include a step of providing a material. In some embodiments, a material is or comprises polymers or proteins. In some embodiments, materials are natural or synthetic. In some embodiments, materials are or comprise agarose, alginate, amyloid, cellulose, chitin, chitosan, collagen, elastin, gelatin, hyaluronic acid, polydimethylsiloxane, poly(ethylene glycol), polyhydroxyalkanoates, poly(lactide-co-glycolide), poly(methyl methacrylate), poly(vinyl-alcohol) (PVA), pullan, resilin, silk, starch, or combinations thereof.

In some embodiments, a step of providing a material can include preparing a material with varying properties, including, but not limited to its: absorbance, compressive strength, compressive modulus, degradation rate, elasticity, morphology, polymer/protein molecular weight, pore size, solution concentration, storage modulus, tangent modulus, or combinations thereof.

In some embodiments, the present disclosure encompasses a recognition that silk fibroin is particularly useful in the context of the present disclosure.

In some embodiments, a scaffold material form is a hydrogel. Scaffolds formed from silk fibroin-based hydrogels possess unique and beneficial characteristics.

Scaffolds formed from such hydrogel materials permit precise and facile introduction of cavity structures as disclosed herein.

In some embodiments, methods and technologies disclosed herein allow for precise control over a wide range of properties of cavities in such materials, including, but not limited to, diameter, spacing between any one or more cavities, morphology of a cavity wall that is formed within a material, thickness of a cavity wall that is formed within a material, loading density of an agent introduced on a cavity wall or within a cavity, and any combinations thereof.

In some embodiments, provided methods and technologies include a step of irradiating a material with a beam of electromagnetic radiation. In some embodiments, a beam of radiation is optical (i.e. a beam of radiation that is in the visible spectrum). In some embodiments, a beam of radiation is ultraviolet. In some embodiments, a beam of radiation is infrared.

In some embodiments, a step of irradiating may either remove or shape a material.

In some embodiments, a step of irradiating is characterized by its wavelength. In some embodiments, a wavelength of a beam is about 80 nm to about 1000 μm. In some embodiments, a wavelength of a beam is about 200 nm to about 1250 nm. In some embodiments, a wavelength of a beam is about 300 nm to about 900 nm.

In some embodiments, a material is transparent to a beam of radiation.

In some embodiments, a beam of radiation is focused to a focal spot. In some embodiments, a focal spot is a diffuse area around a focal point of a beam. In some embodiments, a material is locally heated at a focal spot of a beam. In some embodiments, a material is changed by exposure to electromagnetic radiation at its focal point. In some embodiments, a material is removed when exposed to electromagnetic radiation at its focal point.

In some embodiments, a step of irradiating is characterized in that it is as a pulsed beam. In some embodiments, a pulse beam is characterized in that it has a short pulse duration. In some embodiments, a pulse duration is on the order of femtoseconds.

In some embodiments, a step of irradiating is characterized in that it is as a pulsed beam. In some embodiments, a pulsed beam has a frequency of about 1 Hz to about 100 GHz. In some embodiments, a pulsed beam has a frequency of about 50 MHz. to about 100 MHz.

In some embodiments, a step of irradiating is characterized in that it is as a pulsed beam having an energy. In some embodiments, a pulse energy is less than 1 nJ. In some embodiments, a pulse energy is about 0.5 nJ to about 1 μJ.

In some embodiments, a step of irradiating includes single or multiphoton absorption.

In some embodiments, multiphoton absorption includes absorption of at least two photons. In some embodiments, irradiating by a multiphoton absorption process includes irradiating so that heat from a single photon does not dissipate before another photon hits.

In some embodiments, when initiating a multiphoton absorption, a hydrogel is disrupted. In some embodiments, disrupted hydrogel is removed forming a cavity beneath the surface of the hydrogel.

In some embodiments, a beam of radiation is focused to a point beneath a surface of a material. In some embodiments, a beam of radiation is focused to a point about 1 μm to about 10 mm beneath a surface of a material.

In some embodiments, a beam of radiation forms a cavity characterized by its diameter or size. In some embodiments, a diameter is about 1 μm to about 10 mm.

In some embodiments, cavities are characterized by their cross-sectional size. In some embodiments, a cross-sectional size of a cavity is about 1 μm to about 10 mm.

In some embodiments, a beam of radiation is manipulated so that a focal spot moves beneath a surface of a material to form a three-dimensional cavity.

In some embodiments, a beam of radiation is manipulated so that a focal spot moves beneath a surface of a material to form a three-dimensional cavity according to a shape or pattern. In some embodiments, a three-dimensional shape or pattern is formed within a material and beneath its surface.

In some point, manipulating a focal spot includes moving or shifting a focal spot. In some embodiments, when moving or shifting a focal spot, a cavity formed in a material. In some embodiments, when moving or shifting a focal spot, a cavity extended through a material.

In some embodiments, shifting a position of the focused beam extend the at least one cavity from an initial focal spot so that the hydrogel is characterized by a cavity having a shape or pattern beneath the surface of the hydrogel.

In some embodiments, a step of shifting occurs by moving a focal spot in a direction that is lateral, longitudinal, or normal relative to a surface of the hydrogel.

In some embodiments, a focal spot shifts at a rate of less than about a mm/sec to about several mm/sec. In some embodiments, a focal spot shifts at a rate of about 0.05 mm/sec to about 10 mm/sec.

In some embodiments, lithography may be used to mask certain regions of a material. In some embodiments, masking prevents electromagnetic radiation from passing. In some embodiments, masking includes traditional lithography methods and materials.

In some embodiments, provided methods and technologies form cavities according to a pattern. In some embodiments, a pattern is formed or defined by a scaffold's application or use.

In some embodiments, provided methods and technologies form cavities. In some embodiments, a cavity is formed according to a pattern. In some embodiments, a pattern is defined by a use or application of a scaffold. In some embodiments, for example, a pattern is for a specific tissue regeneration. In some embodiments, for example, a pattern is for a specific sensor. In some embodiments, depending on the application, cavities are referred to as channels, conduits, microchannels, microvasculature, openings, tunnels, vasculature, or voids within a scaffold.

In some embodiments, methods and technologies include producing a porous scaffold that can be used for tissue or tissue engineering. In some embodiments, provided methods and technologies form scaffolds for example resembling tissues or organs. In some embodiments, scaffolds resembling tissues or organs have cavities that form or mimic that of a circulatory system of such tissues or organs. In some embodiments, provided methods and technologies include vascularizing an engineered tissue construct.

In some embodiments, scaffold materials include cells and/or additives, agents, or functional moieties added thereto. In some embodiments, agents include biologically active molecules or macromolecules, cells, drugs, dyes, fluorescent markers, nucleic acids, organic compounds, proteins, etc.

In some embodiments, a step of providing a material includes steps of encapsulating additives, agents, or functional moieties within a material prior to a step of irradiating. In some embodiments, a step of providing a material includes steps of encapsulating cells within a material prior to a step of irradiating. In some embodiments, a step of providing includes steps of encapsulating more than one additives, agents, or functional moieties within a material prior to a step of irradiating. In some embodiments, a step of providing includes steps of encapsulating cells of more than one cell type.

In some embodiments, a step of providing a material includes steps of coating cells and/or additives, agents, or functional moieties on a surface of a material prior to a step of irradiating.

In some embodiments, provided methods of encapsulating include seeding a material with cells and/or additives, agents, or functional moieties. In some embodiments, provided methods include uniformly seeding a material with cells and/or additives, agents, or functional moieties throughout such a material. In some embodiments, provided methods include a step of seeding a material with cells and/or additives, agents, or functional moieties according to a grade. In some embodiments, a grade is from highest density to lowest density.

In some embodiments, provided methods and technologies include irradiating a material including cells and/or additives, agents, or functional moieties.

In some embodiments, when viable cells encapsulated within a material are irradiated the cells remain viable. In some embodiments, when viable cells encapsulated within a material are irradiated, such cells are undamaged. In some embodiments, when additives, agents, and/or functional moieties encapsulated within a material are irradiated, such additives, agents, and/or functional moieties are undamaged.

In some embodiments, provided methods further include steps of depositing removed material. In some embodiments, when removing material, removed material is deposited on an interior surface of a cavity.

In some embodiments, after a step of irradiating, provided methods and technologies include adding agents to an interior surface of a formed cavity.

In some embodiments, cells, additives, agents, and/or functional moieties are transparent to a beam of radiation. In some embodiments, when irradiating a material a beam of radiation does not alter a material, cells, additives, agents, and/or functional moieties.

Among other things, the present disclosure provides scaffolds. In some embodiments, scaffolds include biomechanical scaffolds.

Provided scaffolds are characterized by unique features that provide advantages over existing structures. In particular, provided scaffolds include cavities formed beneath their surface.

In some embodiments, cavities formed beneath a scaffold surface such that provided scaffolds are capable of supporting and sustaining cells and cellular activity. Additionally, in some embodiments, scaffolds as provided herein are biodegradable, biocompatible, cytocompatible, and able to stabilize biologically labile compounds, such as cells, nucleic acids, enzymes.

In some embodiments, scaffolds are or include polymers or proteins. In some embodiments, scaffolds are natural or synthetic. In some embodiments, scaffolds are or include agarose, alginate, amyloid, cellulose, chitin, chitosan, collagen, elastin, gelatin, hyaluronic acid, polydimethylsiloxane, poly(ethylene glycol), polyhydroxyalkanoates, poly(lactide-co-glycolide), poly(methyl methacrylate), poly(vinyl-alcohol) (PVA), pullan, resilin, silk, starch, or combinations thereof.

In some embodiments, provided scaffolds are particularly suitable as soft biomaterials that characterized by physical and mechanical properties that are compatible to match a broad range of human tissues.

In some embodiments, provided scaffolds are non-toxic.

In some embodiments, provided scaffolds are characterized in that they are capable of incorporating additives, agents, or functional moieties that provide or maintain cellular and tissue function. In some embodiments, additives, agents, or functional moieties include any biologically or pharmaceutically active compound. In some embodiments, for example additives, agents, or functional moieties may include, but are not limited to, peptides, antibodies, DNA, RNA, modified RNA/protein composites, glycogens or other sugars, and alcohols.

In some embodiments, provided scaffolds are characterized in that they are transparent to wavelengths in the electromagnetic spectrum. In some embodiments, provided scaffolds are characterized in that they are transparent to at least one wavelength range in the electromagnetic spectrum. In some embodiments, scaffold are transparent from about 200 μm to about 1750 μm. In some embodiments, provided scaffolds are characterized in that they are transparent to at least one wavelength in the electromagnetic spectrum. In some embodiments, provided scaffolds are characterized in that they are transparent throughout.

In some embodiments, scaffolds include cavities.

In some embodiments, cavities are or include channels, conduits, microchannels, microvasculature, openings, tunnels, vasculature, or voids. In some embodiments, cavities are at a depth of at least 200 μm beneath a surface of provided scaffolds.

In some embodiments, cavities are characterized by their diameter or size. In some embodiments, a diameter is about 1 μm to about 10 mm. In some embodiments, cavities are characterized by their dimensional cross-section. In some embodiments, cross-sectional size is about 1 μm to about 10 mm.

In some embodiments, cavities are characterized by their lateral resolution. In some embodiments, cavities have a lateral resolution of less than about 1 μm.

In some embodiments, cavities are characterized in that they extend through provided scaffolds according to at least one shape or pattern.

In some embodiments, a shape or pattern is formed or defined by a scaffold's application or use. In some embodiments, for example, a pattern is for a specific tissue regeneration. In some embodiments, for example, as provided above, a pattern is for a specific sensor. In some embodiments, depending on the application, cavities are referred to as channels, conduits, microchannels, microvasculature, openings, tunnels, vasculature, or voids within a scaffold.

In some embodiments, provided scaffolds are characterized in that they are capable of supporting cells and cell growth. In some embodiments, cells are coated on an outer surface of a scaffold. In some embodiments, cells are encapsulated with a scaffold. In some embodiments, cavities are characterized in that they sustain cells encapsulated or present within such scaffolds.

In some embodiments, cavities are characterized in that their interior walls include material that was removed when the cavity was formed. In some embodiments, cavities allow for infusion of cells within a scaffold.

In some embodiments, cavities formed or patterned within a cell encapsulated scaffold provide access for transporting oxygen and nutrients within a scaffold to cells. In some embodiments, at least one cavity, channel, conduit, opening, tunnel, vasculature, or void within a diffusion limit can be useful for minimizing or reducing the chance of necrosis.

In some embodiments, a predetermined shape or pattern of a cavity includes spacing in a manner that results in formation of more than one cavity being spatially located from another within a diffusion limit. In some embodiments, more than one cavity located within a diffusion limit can be useful for minimizing or reducing the chance of necrosis.

In some embodiments, a pre-determined shape or pattern of such cavities can include spacing at least two of the neighboring elongated structures at a distance in a range of about 100 μm to about 2000 μm.

In some embodiments, cavities support confluent endothelialization. In some embodiments, confluent endothelialization is desirable for vascularization of a tissue construct, and that the resulting scaffold can allow for controlled localization of at least two different cell types within the scaffold.

In some embodiments, provided scaffolds are or include a silk fibroin hydrogel. In some embodiments, the present disclosure encompasses a recognition that silk fibroin hydrogels are a particularly useful because they permit precise and facile introduction of cavity structures.

In some embodiments, scaffolds made of hydrogels are characterized in that they are transparent in the electromagnetic spectrum. In some embodiments, scaffolds made of hydrogels are characterized in that they are transparent to at least one wavelength range the electromagnetic spectrum.

In some embodiments, provided scaffolds can be used, for example, as a scaffold for a engineered tissue or as a drug delivery device. In some embodiments, provided scaffolds can be adjusted to mimic a gradient of cellular densities found in natural tissues or organs. In some embodiments, provided scaffolds can be used for a range of tissue and/or organ engineering applications.

In some embodiments, a material is optically transparent to at least one wavelength in the electromagnetic spectrum. In some embodiments, a material is optically transparent to a range of wavelengths in the electromagnetic spectrum. In some embodiments, a material is optically transparent to at least one wavelength in the visible spectrum. In some embodiments, scaffolds are optically clear from about 200 μm to about 1750 μm.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1 shows an overview of the micromachining process. FIG. 1 at panel (A) shows a schematic of the multiphoton micromachining workstation. FIG. 1 at panel (A in the inset) shows a photograph of the transparent silk hydrogel. FIG. 1 at panel (B) shows a three-dimensional AFM image of one of the lines in FIG. 1 at panel (D). FIG. 1 at panel (C) shows a graph relating line dimensions with pulse energy. Error bars represent 1 SD (n=4). FIG. 1 at panel (D) shows a microphotograph of lines machined into the upper surface of a silk gel at pulse energies ranging from 0.25 nJ (FIG. 1 at panel (D) at the Bottom line) to 5 nJ (FIG. 1 at panel (D) at the Top line). FIG. 1 at (E) End-on view of 30-μm-wide lines machined into a silk gel. Light was incident from the bottom of the image. Ruler on right side measures depth from the surface of the sample. Due to the large area involved, this image was stitched together from a series of microphotographs. FIG. 1 at (E in the inset) shows detail of the cross-section of one line.

FIG. 2 shows a linear absorption spectrum of a silk gel with background subtracted. The vertical line at 810 nm indicates the wavelength used for multiphoton micromachining. The vertical Pump/2 and Pump/3 lines indicate the wavelengths associated with a two-photon and three-photon absorption process, respectively. Linear absorption is driven by tyrosine and tryptophan residues in the silk. Spectrum was filtered by a five-point moving average to reduce noise.

FIG. 3 shows Results from the knife edge measurement of the laser spot size. FIG. 3 at the top panel shows results in the X direction. FIG. 3 at the bottom panel shows results from the Y direction. Propagation was in the Z direction. The full width at half maximum spot size was calculated to be 5 μm in the X direction and 6 μm in the Y direction.

FIG. 4 shows light incident from the left portion of the image was used to micromachine features at various depths in a gel. Each feature was made by a single pass of the laser scanning into the page at ˜10 nJ per pulse of power and a rate of 75 μm/s. Large boxes show a zoomed-in view of the outlined area around each individual feature. The shape of each feature remains constant at each depth, indicating that self-focusing is not significantly deflecting the beam in the material. Due to the large area involved, this image was stitched together from several different photomicrographs.

FIG. 5 shows amplified laser pulses incident from the bottom were used to create voids in the silk hydrogels at various pulse energies. Symmetrical features using 1-μJ pulses FIG. 5 at left panel indicate that self-focusing is negligible. Strong self-focusing effects are evident in the asymmetrical shape from 10-μJ pulses (FIG. 5 at the Center panel) and 20-μJ (FIG. 5 at the Right panel) pulse energies. All features were made by a single pulse at each location.

FIG. 6 shows an overview of two test patterns machined into the gel. FIG. 6 at panel (A) shows a three-dimensional model of a helical pattern input into the control program. FIG. 6 at panel (B) shows a confocal microscope image of a cross-section of the helix showing the machined region in black. (Scale bar, 100 μm). FIG. 6 at panel (C) shows a reslice of the confocal stack along the dashed line in FIG. 6 at panel (B). (Scale bar, 100 μm). FIG. 6 at panel (D) shows a three-dimensional reconstruction of the segmented confocal data showing the machined feature. FIG. 6 at panel (E) shows a three-dimensional model of a branching pattern input into the machining control program. FIG. 6 at panel (F) shows a three-dimensional reconstruction of resulting machined region made by segmenting the confocal images. FIG. 6 at panel (G) shows a confocal slice showing a cross-section of the micromachined region. (Scale bar, 100 μm). FIG. 6 at panel (H) shows cross-sections of the confocal volumes at the indicated lines. (Scale bar, 100 μm). FIG. 6 at panel (I) shows cross-sections of the confocal volumes at the indicated lines. (Scale bar, 100 μm).

FIG. 7 at the left panel shows a confocal cross-section of the vascular-like pattern described in FIG. 6 using only the autofluorescence of silk fibroin for contrast. An increase in autofluorescence can be appreciated immediately surrounding the machined region which suggests that an increased density of silk is present in those locations. FIG. 7 at the top/bottom right panels are reslices of the confocal stack along the indicated lines. (Scale bar applies to all panels).

FIG. 8 shows micromachined features in vitro. FIG. 8 at panel (A) shows machined lines on the surface of a gel at day 1. FIG. 8 at panel (B) shows machined lines on the surface of a gel at day 3. FIG. 8 at panel (C) shows machined lines on the surface of a gel at day 5. FIG. 8 at panel (D) shows machined lines on the surface of a gel at day 8. Arrows indicate cells growing along the machined lines. FIG. 8 at panel (D) shows a fluorescently labeled cells growing in the lines. (Scale bar, 100 μm long). FIG. 8 at panel (D) shows a slightly different region of the gel as high cell density obscured the features at the location of the other images. FIG. 8 at panel (E) shows a cartoon of micromachining of a gel laden with hMSCs. FIG. 8 at panel (E and the Inset) shows a bright-field image of the machined region. (Scale bar, 250 μm). FIG. 8 at panel (F) shows confocal images of the cell-laden gel following live/dead staining 76 μm below the plane of machining. FIG. 8 at panel (G) shows confocal images of the cell-laden gel following live/dead staining 62 μm above the plane of machining. FIG. 8 at panel (H) shows confocal images of the cell-laden gel following live/dead staining in the plane of machining. Dashed lines outline the micromachined region. (Scale bar, 250 μm). FIG. 8 at panel (I) shows a close-up of living cells irradiated by the beam above the focal plane. FIG. 8 at panel (J) shows a close-up of living cells irradiated by the beam below the focal plane.

FIG. 9 shows cell infiltration into machined features. FIG. 9 at panel (A at the Top) shows a three-dimensional model of the pattern machined into hydrogels that were subsequently seeded with cells in vitro. FIG. 9 at panel (A at the Bottom) shows a series of confocal images of fibroblasts growing within a Y-shaped machined feature on day 9 after seeding. Each image is separated by 10 μm in the Z direction. (Scale bar, 100 μm). FIG. 9 at panel (B at the Top) shows a three-dimensional model of the pattern machined into a hydrogel that was subsequently implanted sub cutaneous in mice. Lines marked “(i-iii)” indicate the confocal cross-sections shown in FIG. 9 at panel (B at the Bottom). The white circles in FIG. 9 at panel (B at the Bottom, (i)) and FIG. 9 at panel (B at the Bottom, (ii)) correspond to the main branch diameter and approximate location in the construct. The smaller circles in FIG. 9 at panel (B at the Bottom, (iii)) correspond to the secondary branch diameters. Cells had infiltrated to the bottom of the main branch FIG. 9 at panel (B at the Bottom, (ii)) and had begun extending down one of the secondary branches FIG. 9 at panel (B at the bottom, (iii)). (All scale bars, 100 μm).

FIG. 10 shows confocal images of micromachined gel containing a feature with main branch diameter of 400 μm. FIG. 10 at panel (A) shows a feature on the surface of the gel. FIG. 10 at panel (B) shows a feature 74 μm below the surface of the gel. Dashed white lines indicate the diameter and approximate location of the main branch of the feature. No cells are present in the micromachined region on the surface, but are found in high concentrations at the bottom of the main branch. This sample was recovered 3 wk after implantation.

FIG. 11 shows confocal images of micromachined gel containing a feature with main branch diameter of 200 μm. FIG. 11 at panel (A) shows the top surface of the gel. FIG. 10 at panel (B) shows 50.5 μm below the surface, and FIG. 10 at panel (C) shows C is 70 μm below the surface of the gel. A relative absence of cells can be seen at the surface whereas cells conforming to the shape of the feature are visible below the surface of the gel. This sample was recovered 3 wk after implantation. (Scale bar, 100 μm).

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration”: As used herein, the term “administration” refers to the administration of a composition to a subject. Administration may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal and vitreal.

“Agent”: As used herein, the term “agent” may refer to a compound or entity of any chemical class including, for example, polypeptides, nucleic acids, saccharides, lipids, small molecules, metals, or combinations thereof. As will be clear from context, in some embodiments, an agent can be or include a cell or organism, or a fraction, extract, or component thereof. In some embodiments, an agent is agent is or includes a natural product in that it is found in and/or is obtained from nature. In some embodiments, an agent is or includes one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or includes a polymer. In some embodiments, an agent is not a polymer and/or is substantially free of any polymer. In some embodiments, an agent contains at least one polymeric moiety. In some embodiments, an agent lacks or is substantially free of any polymeric moiety.

“Amino acid”: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

“Antibody”: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. Amino acid sequence comparisons among antibody polypeptide chains have defined two light chain (κ and λ) classes, several heavy chain (e.g., μ, γ, α, ε, δ) classes, and certain heavy chain subclasses (α1, α2, γ1, γ2, γ3, and γ4). Antibody classes (IgA [including IgA1, IgA2], IgD, IgE, IgG [including IgG1, IgG2, IgG3, IgG4], IgM) are defined based on the class of the utilized heavy chain sequences. For purposes of the present disclosure, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is monoclonal; in some embodiments, an antibody is monoclonal. In some embodiments, an antibody has constant region sequences that are characteristic of mouse, rabbit, primate, or human antibodies. In some embodiments, an antibody sequence elements are humanized, primatized, chimeric, etc., as is known in the art. Moreover, the term “antibody” as used herein, will be understood to encompass (unless otherwise stated or clear from context) can refer in appropriate embodiments to any of the art-known or developed constructs or formats for capturing antibody structural and functional features in alternative presentation. For example, in some embodiments, the term can refer to bi- or other multi-specific (e.g., zybodies, etc.) antibodies, Small Modular ImmunoPharmaceuticals (“SMIPs™”), single chain antibodies, cameloid antibodies, and/or antibody fragments. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc]

“Associated” or “Associated with”: As used herein, the term “associated” or “associated with” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Binding”: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

“Binding agent”: In general, the term “binding agent” is used herein to refer to any entity that binds to a target of interest as described herein. In many embodiments, a binding agent of interest is one that binds specifically with its target in that it discriminates its target from other potential bidning partners in a particular interaction contect. In general, a binding agent may be or include an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may include a “generic” binding moiety (e.g., one of biotin/avidin/streptaviding and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or include polypeptides (including, e.g., antibodies or antibody fragments). In some embodiments, binding agents are or include small molecules. In some embodiments, binding agents are or include nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or include carbohydrates. In some embodiments, binding agents are or include lectins. In some embodiments, binding agents are or include peptidomimetics. In some embodiments, binding agents are or include scaffold proteins. In some embodiments, binding agents are or include mimeotopes. In some embodiments, binding agents are or include stapled peptides. In certain embodiments, binding agents are or include nucleic acids, such as DNA or RNA.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Biologically active”: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

“Characteristic portion”: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

“Comparable”: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

“Corresponding to”: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190th residue in the first polymer but rather corresponds to the residue found at the 190th position in the reference polymer; those of ordinary skill in the art readily appreciate how to identify “corresponding” amino acids, including through use of one or more commercially-available algorithms specifically designed for polymer sequence comparisons.

“Detection entity”: The term “detection entity” as used herein refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu ¹⁸⁷Re, ¹¹¹In, ⁹⁰Y, ^{99m}Tc, ¹⁷⁷Lu, ⁸⁹Zr etc.) fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

“Determine”: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

“Encapsulated”: The term “encapsulated” is used herein to refer to substances that are completely surrounded by another material.

“Functional”: As used herein, a “functional” biological molecule is a biological molecule in a form in which it exhibits a property and/or activity by which it is characterized. A biological molecule may have two functions (i.e., bi-functional) or many functions (i.e., multifunctional).

“Graft rejection”: The term “graft rejection” as used herein, refers to rejection of tissue transplanted from a donor individual to a recipient individual. In some embodiments, graft rejection refers to an allograft rejection, wherein the donor individual and recipient individual are of the same species. Typically, allograft rejection occurs when the donor tissue carries an alloantigen against which the recipient immune system mounts a rejection response.

“High Molecular Weight Polymer”: As used herein, the term “high molecular weight polymer” refers to polymers and/or polymer solutions comprised of polymers (e.g., protein polymers, such as silk) having molecular weights of at least about 200 kDa, and wherein no more than 30% of the silk fibroin has a molecular weight of less than 100 kDa. In some embodiments, high molecular weight polymers and/or polymer solutions have an average molecular weight of at least about 100 kDa or more, including, e.g., at least about 150 kDa, at least about 200 kDa, at least about 250 kDa, at least about 300 kDa, at least about 350 kDa or more. In some embodiments, high molecular weight polymers have a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa.

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Identity”: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Low Molecular Weight Polymer”: As used herein, the term “low molecular weight polymer” refers to polymers and/or polymer solutions, such as silk, comprised of polymers (e.g., protein polymers) having molecular weights within the range of about 20 kDa-about 400 kDa. In some embodiments, low molecular weight polymers (e.g., protein polymers) have molecular weights within a range between a lower bound (e.g., about 20 kDa, about 30 kDa, about 40 kDa, about 50 kDa, about 60 kDa, or more) and an upper bound (e.g., about 400 kDa, about 375 kDa, about 350 kDa, about 325 kDa, about 300 kDa, or less). In some embodiments, low molecular weight polymers (e.g., protein polymers such as silk) are substantially free of, polymers having a molecular weight above about 400 kD. In some embodiments, the highest molecular weight polymers in provided scaffolds are less than about 300-about 400 kD (e.g., less than about 400 kD, less than about 375 kD, less than about 350 kD, less than about 325 kD, less than about 300 kD, etc). In some embodiments, a low molecular weight polymer and/or polymer solution can include a population of polymer fragments having a range of molecular weights, characterized in that: no more than 15% of the total moles of polymer fragments in the population has a molecular weight exceeding 200 kDa, and at least 50% of the total moles of the silk fibroin fragments in the population has a molecular weight within a specified range, wherein the specified range is between about 3.5 kDa and about 120 kDa or between about 5 kDa and about 125 kDa.

“Marker”: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present disclosure a useful marker need not distinguish tumors of a particular subclass with 100% accuracy.

“Modulator”: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

“Nanoparticle”: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they include an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

“Nanoparticle composition”: As used herein, the term “nanoparticle composition” refers to a composition that contains at least one nanoparticle and at least one additional agent or ingredient. In some embodiments, a nanoparticle composition contains a substantially uniform collection of nanoparticles as described herein.

“Nucleic acid”: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present disclosure. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can include nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment includes at least 3, 4, 5, 6, 7, 8, 9, 10, or more residues. In some embodiments, a nucleic acid is or includes natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present disclosure is specifically directed to “unmodified nucleic acids,” meaning nucleic acids (e.g., polynucleotides and residues, including nucleotides and/or nucleosides) that have not been chemically modified in order to facilitate or achieve delivery.

“Pharmaceutical composition”: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., atmospheric pressure of about 1. In some embodiments, physiological conditions utilize or include an aqueous environment (e.g., water, saline, Ringers solution, or other buffered solution); in some such embodiments, the aqueous environment is or includes a phosphate buffered solution (e.g., phosphate-buffered saline).

“Polypeptide”: The term “polypeptide”, as used herein, generally has its art-recognized meaning of a polymer of at least three amino acids, linked to one another by peptide bonds. In some embodiments, the term is used to refer to specific functional classes of polypeptides. For each such class, the present specification provides several examples of amino acid sequences of known exemplary polypeptides within the class; in some embodiments, such known polypeptides are reference polypeptides for the class. In such embodiments, the term “polypeptide” refers to any member of the class that shows significant sequence homology or identity with a relevant reference polypeptide. In many embodiments, such member also shares significant activity with the reference polypeptide. Alternatively or additionally, in many embodiments, such member also shares a particular characteristic sequence element with the reference polypeptide (and/or with other polypeptides within the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or include a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, a polypeptide may include natural amino acids, non-natural amino acids, or both. In some embodiments, a polypeptide may include only natural amino acids or only non-natural amino acids. In some embodiments, a polypeptide may include D-amino acids, L-amino acids, or both. In some embodiments, a polypeptide may include only D-amino acids. In some embodiments, a polypeptide may include only L-amino acids. In some embodiments, a polypeptide may include one or more pendant groups, e.g., modifying or attached to one or more amino acid side chains, and/or at the polypeptide's N-terminus, the polypeptide's C-terminus, or both. In some embodiments, a polypeptide may be cyclic. In some embodiments, a polypeptide is not cyclic. In some embodiments, a polypeptide is linear.

“Polysaccharide”: The term “polysaccharide” refers to a polymer of sugars. Typically, a polysaccharide includes at least three sugars. In some embodiments, a polypeptide includes natural sugars (e.g., glucose, fructose, galactose, mannose, arabinose, ribose, and xylose); alternatively or additionally, in some embodiments, a polypeptide includes one or more non-natural amino acids (e.g. modified sugars such as 2′-fluororibose, 2′-deoxyribose, and hexose).

“Porosity”: The term “porosity” as used herein, refers to a measure of void spaces in a material and is a fraction of volume of voids over the total volume, as a percentage between 0 and 100%. A determination of a porosity is known to a skilled artisan using standardized techniques, for example mercury porosimetry and gas adsorption (e.g., nitrogen adsorption).

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may include natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Reference”: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not include a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., includes at least one detectable moiety). In some embodiments, a small molecule is a therapeutic. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered acceptable for use in accordance with the present application.

“Solution”: As used herein, the term “solution” broadly refers to a homogeneous mixture composed of one phase. Typically, a solution includes a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Stable”: The term “stable,” when applied to compositions herein, means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Sustained release”: The term “sustained release” is used herein in accordance with its art-understood meaning of release that occurs over an extended period of time. The extended period of time can be at least about 3 days, about 5 days, about 7 days, about 10 days, about 15 days, about 30 days, about 1 month, about 2 months, about 3 months, about 6 months, or even about 1 year. In some embodiments, sustained release is substantially burst-free. In some embodiments, sustained release involves steady release over the extended period of time, so that the rate of release does not vary over the extended period of time more than about 5%, about 10%, about 15%, about 20%, about 30%, about 40% or about 50%. In some embodiments, sustained release involves release with first-order kinetics. In some embodiments, sustained release involves an initial burst, followed by a period of steady release. In some embodiments, sustained release does not involve an initial burst. In some embodiments, sustained release is substantially burst-free release.

“Therapeutic agent”: As used herein, the phrase “therapeutic agent” refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

“Therapeutically effective amount”: As used herein, the term “therapeutically effective amount” means an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” To give but one example, a refractory subject may have a low bioavailability such that clinical efficacy is not obtainable. In some embodiments, reference to a therapeutically effective amount may be a reference to an amount as measured in one or more specific tissues (e.g., a tissue affected by the disease, disorder or condition) or fluids (e.g., blood, saliva, serum, sweart, tears, urine, etc). Those of ordinary skill in the art will appreciate that, in some embodiments, a therapeutically effective amount may be formulated and/or administered in a single dose. In some embodiments, a therapeutically effective amount may be formulated and/or administered in a plurality of doses, for example, as part of a dosing regimen.

“Treating”: As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

“Variant”: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

Among other things, the present disclosure provides scaffolds and methods of preparing and using such scaffolds. Various embodiments according to the present disclosure are described in detail herein. In particular, the present disclosure describes scaffolds made of scaffolds and their use in various applications, including, for example: biomaterials, biomedical, biosensing, drug delivery, electronics, optics, optogenetics, photonics, regenerative medicine, tissue engineering applications, tissue regeneration, utility for transparent tissues, and/or tunable degradation and/or controlled release applications.

The ability to controllably shape biomaterials on the microscale in two, and especially three, dimensions is important given the utility of these structures in guiding cellular growth, differentiation, gene expression, and regeneration. (See Lapointe V L S, et al., “Nanoscale Topography and Chemistry Affect Embryonic Stem Cell Self-Renewal and Early Differentiation”, 2 Adv Healthc Mater 12, 1644-1650 (2013); see also Shi X, et al. “Directing Osteogenesis of Stem Cells with Drug-Laden, Polymermicrosphere-Based Micropatterns Generated by Teflon Microfluidic Chips”, 22 Adv Funct Mater 18, 3799-3807 (2012); Jeon O, Alsberg E “Regulation of stem cell fate in a three-dimensional micropatterned dual-crosslinked hydrogel system”, 23 Adv Funct Mater 38, 4765-4775 (2013); Mandal B B, et al., “Cell proliferation and migration in silk fibroin 3D scaffolds”, 30 Biomaterials 15, 2956-2965 (2009). The use of soft, scaffolds, however, poses challenges in fabrication due to their mechanical characteristics. Widely adopted biomaterial microfabrication techniques such as soft- and photolithography are largely limited to two dimensions. The recent advent of 3D printing technology has exploited the interaction of light with materials to rapidly prototype parts for a variety of industries, and has expanded to significantly impact the biomedical field. (See Giannatsis J, et al., Additive Fabrication Technologies Applied to Medicine and Health Care: A review”, 40 Int J Adv Manuf Technol, 116-127 (2007); see also Rengier F, et al., “3D printing based on imaging data: Review of medical applications”, 5 Int J CARS 4, 335-341 (2010); Cohen A, et al., “Mandibular Reconstruction Using Stereolithographic 3-Dimensional Printing Modeling Technology”, 108 Oral Surg Oral Med Oral Pathol Oral Radiol Endod 5, 661-666 (2009)). Microscale 3D printing has also shown promise for tissue engineering and regenerative medicine applications. (See Murphy et al., 3D Bioprinting of Tissues and Organs“, 32 Nat Biotechnol 8, 773-785 (2014); see also Peltola S M, et al., A review of rapid prototyping techniques for tissue engineering purposes” 40 Ann Med 4, 268-280 (2008).

One of the primary obstacles in building critically-sized engineered tissue constructs includes the diffusion limit of oxygen and nutrients. Scaffold constructs that exceed dimensions beyond several hundred micrometers are generally prone to necrosis at the core of the construct and ultimately fail to integrate with host tissue in the long-term due to lack of blood perfusion (Lokmic and Mitchell, 2008; Rouwkema et al., 2008). The natural steps of vascularization within a critically-sized construct do not generally occur within a sufficient time frame to supply the entire construct with necessary oxygen and nutrients. Furthermore, such necrosis can stimulate inflammatory responses in vivo, leading to undesirable outcomes with the implantation of the constructs.

Among other things, the present disclosure presents materials and methods for generating cavities, including: channels, conduits, openings, tunnels, vasculature, or voids as small as 1 μm in diameter within a biocompatible hydrogel using multiphoton absorption (MPA). Furthermore, the present materials are formed and the present methods demonstrate techniques functions in the absence of exogenous photoinitiators or chemical cross-linkers.

### Scaffolds

In some embodiments, provided scaffolds are capable of being formed, molded, shaped, and/or machined into desired structures. In some embodiments, provided scaffolds may for example be formed, molded, shaped, and/or machined as a tissue or organ. In some embodiments, provided scaffolds may for example be formed, molded, shaped, and/or machined as a sensor, such as a biosensor.

Materials

In some embodiments, scaffolds utilized in accordance with the present disclosure are, comprise or consist of polymers. In some embodiments, such polymers are or comprise proteins. In some embodiments, protein polymers of are selected from the group consisting of agarose, alginate, amyloid, cellulose, chitin, chitosan, collagen, elastin, gelatin, hyaluronic acid, pullan, resilin, silk, starch, or combinations thereof. Those skilled in the art will recognize that this is an exemplary, not comprehensive, list of polymers.

In some embodiments, scaffolds as described herein are formed from and/or comprise of scaffolds. In some embodiments, materials are natural or synthetic.

In some embodiments, scaffolds may include one or more other polypeptides or proteins. Such polymers include, for example, certain polyesters, polyanhydrides, polycaptolactone, polyorthoesters, polyphosphazenes, polyphosphoesters, certain polyhydroxyacids, polypropylfumerates, polycaprolactones, polyamides, poly(amino acids), polyacetals, polyethers, biodegradable polycyanoacrylates, biodegradable polyurethanes, poly(glycerol sebacates), elastomeric poly(glycerol sebacates polysaccharides), polypyrrole, polyanilines, polythiophene, polystyrene, polyesters, polyureas, poly(ethylene vinyl acetate), polypropylene, polymethacrylate, polyethylene, polycarbonates, poly(ethylene oxide), polysaccharides, co-polymers, and combinations thereof. For example, specific biodegradable polymers that may be used include but are not limited to polylysine (e.g., poly(L-lysine) (“PLL”)), poly(lactic acid) (“PLA”), poly(glycolic acid) (“PGA”), polylactic acid/poly(glycolide-colactide) copolymer (“PLGA”), poly(caprolactone) (“PCL”), poly(lactide-co-glycolide) (“PLG”), poly(lactide-co-caprolactone) (“PLC”), poly(glycolide-co-caprolactone) (“PGC”), poly(styrene sulfonate) (“SPS”), poly(acrylic acid) (“PAA”), linear poly(ethylene imine) (“LPEI”), poly(diallyldimethyl ammonium chloride) (“PDAC”), and poly(allylamine hydrochloride) (“PAH”), polydimethylsiloxane, poly(ethylene glycol), polyhydroxyalkanoates, poly(methyl methacrylate), or poly(vinyl-alcohol) (PVA). Another exemplary degradable polymer is poly (beta-amino esters), which may be suitable for use in accordance with the present application.

In some embodiments, provided scaffolds are characterized by unique features that provide advantages over existing scaffold materials.

In some embodiments, provided scaffolds are non-toxic.

In some embodiments, provided scaffolds are biocompatible.

In some embodiments, provided scaffolds are biodegradable. In some embodiments, provided scaffolds are programmably degradable.

In some embodiments, provided scaffolds have cavities formed therein. Some examples of cavities depending on the application may include for example: channels, conduits, microvasculature, openings, tunnels, vasculature, or voids. In some embodiments, cavities are formed beneath a surface of a scaffold. In some embodiments, such cavities may be formed several hundred microns beneath a surface of a scaffold.

In some embodiments, cavities are at least several millimeters below a surface of a biomechanical scaffold. In some embodiments, cavities are at least 10 μm below a surface of a biomechanical scaffold. In some embodiments, cavities are at least 20 μm, at least 30 μm, at least 40 μm, at least 50 μm, at least 60 μm, at least 70 μm, at least 80 μm, at least 90 μm, at least 100 μm, at least 110 μm, at least 120 μm, at least 130 μm, at least 140 μm, at least 150 μm, at least 160 μm, at least 170 μm, at least 180 μm, at least 190 μm, at least 200 μm, at least 210 μm, at least 220 μm, at least 230 μm, at least 240 μm, at least 250 μm, at least 260 μm, at least 270 μm, at least 280 μm, at least 290 μm, at least 300 μm, at least 325 μm, at least 350 μm, at least 375 μm, at least 400 μm, at least 425 μm, at least 450 μm, at least 475 μm, at least 500 μm, at least 525 μm, at least 550 μm, at least 575 μm, at least 600 μm, at least 625 μm, at least 650 μm, at least 675 μm, at least 700 μm, at least 725 μm, at least 750 μm, at least 775 μm, at least 800 μm, at least 825 μm, at least 850 μm, at least 875 μm, at least 900 μm, at least 925 μm, at least 950 μm, at least 975 μm, at least 1000 μm, at least 1025 μm, at least 1050 μm, at least 1075 μm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3.0 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, at least 4.0 mm, at least 4.1 mm, at least 4.2 mm, at least 4.3 mm, at least 4.4 mm, at least 4.5 mm, at least 4.6 mm, at least 4.7 mm, at least 4.8 mm, at least 4.9 mm, at least 5.0 mm, at least 6.0 mm, at least 7.0 mm, at least 8.0 mm, at least 9.0 mm, at least 10.0 mm, or more below a surface of a biomechanical scaffold.

In some embodiments, cavities formed within a provided biomechanical scaffold are characterized in that a width and length of such cavities is approximately nano-scale to macro-scale. In some embodiments, provided scaffolds include cavities that have an x-dimension as small as about 1 micron. In some embodiments, provided scaffolds include cavities that have a y-dimension as small as about 1 micron.

In some embodiments, cavities have a diameter of about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 1.3 μm, 1.4 μm, 1.5 μm, 1.6 μm, 1.7 μm, 1.8 μm, 1.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.5 μm, 11.0 μm, 11.5 μm, 12.0 μm, 12.5 μm, 13.0 μm, 13.5 μm, 14.0 μm, 14.5 μm, 15.0 μm, 15.5 μm, 16.0 μm, 16.5 μm, 17.0 μm, 17.5 μm, 18.0 μm, 18.5 μm, 19.0 μm, 19.5 μm, 20 μm, 21 μm, 22 μm, 23 μm, 24 μm, 25 μm, 26 μm, 27 μm, 28 μm, 29 μm, 30 μm, 35 μm, 40 μm, 45 μm, 50 μm, 100 μm, or more.

In some embodiments, cavities have an x-dimension or a y-dimension of about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 1.3 μm, 1.4 μm, 1.5 μm, 1.6 μm, 1.7 μm, 1.8 μm, 1.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.5 μm, 11.0 μm, 11.5 μm, 12.0 μm, 12.5 μm, 13.0 μm, 13.5 μm, 14.0 μm, 14.5 μm, 15.0 μm, 15.5 μm, 16.0 μm, 16.5 μm, 17.0 μm, 17.5 μm, 18.0 μm, 18.5 μm, 19.0 μm, 19.5 μm, 20 μm, 21 μm, 22 μm, 23 μm, 24 μm, 25 μm, 26 μm, 27 μm, 28 μm, 29 μm, 30 μm, 35 μm, 40 μm, 45 μm, 50 μm, 100 μm, or more.

In some embodiments, provided scaffolds exhibit at least some transparency to electromagnetic radiation in a range between about 80 nm to about 1000 μm. In some embodiments, provided scaffolds exhibit transparency to electromagnetic wavelengths between about 200 nm to about 1750 nm.

In some embodiments, provided scaffolds exhibit optical clarity and transparency to wavelengths in the visible spectrum.

In some embodiments, provided scaffolds exhibit transparency to electromagnetic wavelengths above about 200 nm. In some embodiments, provided scaffolds exhibit transparency to electromagnetic wavelengths between 200 nm and 250 nm. In some embodiments, provided scaffolds exhibit transparency to electromagnetic wavelengths between 250 nm and 300 nm. In some embodiments, provided scaffolds exhibit transparency to electromagnetic wavelengths between 300 nm and 350 nm. In some embodiments, provided scaffolds exhibit transparency to electromagnetic wavelengths between 350 nm and 400 nm. In some embodiments, provided scaffolds exhibit transparency to electromagnetic wavelengths between 400 nm and 450 nm. In some embodiments, provided scaffolds exhibit transparency to electromagnetic wavelengths between 450 nm and 500 nm. In some embodiments, provided scaffolds exhibit transparency to electromagnetic wavelengths between 500 nm and 550 nm. In some embodiments, provided scaffolds exhibit transparency to electromagnetic wavelengths between 550 nm and 600 nm. In some embodiments, provided scaffolds exhibit transparency to electromagnetic wavelengths between 600 nm and 650 nm. In some embodiments, provided scaffolds exhibit transparency to electromagnetic wavelengths between 650 nm and 700 nm. In some embodiments, provided scaffolds exhibit transparency to electromagnetic wavelengths between 700 nm and 750 nm. In some embodiments, provided scaffolds exhibit transparency to electromagnetic wavelengths between 750 nm and 800 nm. In some embodiments, provided scaffolds exhibit transparency to electromagnetic wavelengths between 800 nm and 850 nm. In some embodiments, provided scaffolds exhibit transparency to electromagnetic wavelengths between 850 nm and 900 nm. In some embodiments, provided scaffolds exhibit transparency to electromagnetic wavelengths between 900 nm and 950 nm. In some embodiments, provided scaffolds exhibit transparency to electromagnetic wavelengths between 950 nm and 1000 nm. In some embodiments, provided scaffolds exhibit transparency to electromagnetic wavelengths between 1000 nm and 1050 nm. In some embodiments, provided scaffolds exhibit transparency to electromagnetic wavelengths between 1050 nm and 1100 nm. In some embodiments, provided scaffolds exhibit transparency to electromagnetic wavelengths between 1100 nm and 1150 nm. In some embodiments, provided scaffolds exhibit transparency to electromagnetic wavelengths between 1150 nm and 1200 nm. In some embodiments, provided scaffolds exhibit transparency to electromagnetic wavelengths between 1200 nm and 1250 nm. In some embodiments, provided scaffolds exhibit transparency to electromagnetic wavelengths between 1250 nm and 1300 nm. In some embodiments, provided scaffolds exhibit transparency to electromagnetic wavelengths between 1300 nm and 1350 nm. In some embodiments, provided scaffolds exhibit transparency to electromagnetic wavelengths between 1350 nm and 1400 nm. In some embodiments, provided scaffolds exhibit transparency to electromagnetic wavelengths between 1400 nm and 1450 nm. In some embodiments, provided scaffolds exhibit transparency to electromagnetic wavelengths between 1450 nm and 1500 nm. In some embodiments, provided scaffolds exhibit transparency to electromagnetic wavelengths between 1500 nm and 1550 nm. In some embodiments, provided scaffolds exhibit transparency to electromagnetic wavelengths between 1550 nm and 1600 nm. In some embodiments, provided scaffolds exhibit transparency to electromagnetic wavelengths between 1600 nm and 1650 nm. In some embodiments, provided scaffolds exhibit transparency to electromagnetic wavelengths between 1650 nm and 1700 nm. In some embodiments, provided scaffolds exhibit transparency to electromagnetic wavelengths between 1700 nm and 1750 nm.

Uses of Scaffolds

Provided scaffolds offer new opportunities at the intersection of biology and technology. Provided scaffolds can be valuably employed providing a new format of silk fibroin with beneficial attributes, for example, optical, mechanical, and/or structural properties. Scaffolds as provided herein are therefore particularly suitable as soft biomaterials characterized by physical and mechanical properties that are tunable to match a broad range of human tissues, for example from nerves to cartilage, by mimicking the hydrated nature of the extracellular space. Indeed, the ability to combine transparency with the well-established, tunable biophysical, biochemical and biological properties of scaffolds shines a new light on hydrogels, enabling the engineering of highly tunable tissue-equivalent constructs with enhanced optical and photonic functionalities.

In some embodiments, provided scaffolds are useful for example in cells growth, for cell regeneration, as an organ or organ replacement, as a sensor or biosensor, or as tissues.

In some embodiments, scaffolds as described herein are useful for cell growth or cell regeneration. In some embodiments, provided scaffolds having a cavity formed within it provide a location for cells to seed. In some embodiments, such scaffolds provide support for cellular activity. In some embodiments, cavities within a biomechanical scaffold provide access for oxygen and nutrients to cells within scaffold.

In some embodiments, scaffolds as provided herein are ideally suited for cellular growth and/or cell implantation. In some embodiments, scaffolds formed from methods and technologies as provided herein are ideally suited for cellular growth and/or cell implantation. In some embodiments, scaffolds of the present disclosure are characterized by mechanical properties that are particularly suitable for use in supporting cell growth, function, viability, and/or differentiation.

In some embodiments, cells are encapsulated within or coated on a surface of a scaffold. In some embodiments, cavities are formed within a scaffold including cells coated, implanted or encapsulated within a scaffold. In some embodiments, cavities formed within these materials result in vasculature. In some embodiments, vasculature provides access for nutrients, oxygen and removal of waste from these cells.

In some embodiments, provided scaffolds are characterized in that they are capable of being encapsulated with cells. In some embodiments, provided scaffolds are encapsulated with cells after formation of cavities therein.

In some embodiments, provided scaffolds with cavities formed within provide a surface for cell adhesion. In some embodiments, cells are coated or seeded onto a surface of provided cavities. In some embodiments, a surface of a cavity provided within a scaffold provides a surface for cell adhesion.

In some embodiments, when hydrogel material is removed from to form a cavity within a provided scaffold, removed material is deposited on a surface a cavity. In such embodiments, deposited hydrogel material provides a surface for cell adhesion.

In some embodiments, provided scaffolds are encapsulated with cells prior to formation of cavities within its structure. In some embodiments, cells are uniformly encapsulated throughout a scaffold. In some embodiments, cells are uniformly coated on a scaffold. In some embodiments, cells are uniformly encapsulated within a scaffold. In some embodiments, cells are uniformly encapsulated throughout a hydrogel. In some embodiments, cells are coated, encapsulated or seeded according to a pattern or gradient such that the cell density is not uniform throughout such a scaffold.

In some embodiments, scaffolds as disclosed herein with cavities formed beneath it surface can support cell growth such that they allow cells to penetrate deep within the scaffold.

In some embodiments, cells include multiple different cells or cell types. In some embodiments, scaffolds are arranged and constructed to support different types of cells.

In some embodiments, scaffolds are arranged and constructed for cell differentiation.

In some embodiments, cavities are formed within materials including cells implanted, encapsulated, or coated on a surface of a material. In some embodiments, cavities formed within these materials result in vasculature that mimics that of natural tissues and organs. In some embodiments, vasculature provides access for nutrients, oxygen and removal of waste from these cells and tissues.

In some embodiments, provided methods and technologies are useful to form tissues or organs from materials with cells implanted therein. In some embodiments, additives, agents or functional moieties may be added to scaffold to support cell and/or tissue growth.

In some embodiments, scaffolds as described herein are useful as tissues, for tissue regeneration, or as a replacement for a tissue or organ in a subject. In some embodiments, scaffolds as disclosed herein are useful for cultivating tissues and/or organs. In some embodiments, provided scaffolds may be useful for example as tissue or an organ. In some embodiments, provided scaffolds having cavities formed therein provide access for oxygen and nutrients to cells within scaffold thereby supporting such tissues or organs.

In some embodiments, scaffolds as described herein are useful in regenerative medicine for transparent tissue scaffolds. In some embodiments, scaffolds are characterized in that they are capable of being encapsulated, seeded and/or functionalized. In some embodiments, scaffolds are characterized in that they are capable of being encapsulated, seeded and/or functionalized with cells, for example human cornea epithelial cells (HCECs). In some embodiments, scaffolds are characterized in that when encapsulated or seeded with cells they are capable of maintaining cell viability and proliferation over a period with no appreciable difference when compared to other hydrogels, such a collagen hydrogels. In some embodiments, scaffolds are characterized in that when encapsulated with cells they are capable of maintaining cell viability and proliferation over a period of between 14 and 28 days.

In some embodiments, scaffolds are characterized in that when encapsulated or seeded cells would culture on a hydrogel surface. In some embodiments, cells would remain viable for a period up to 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, or 65 days.

In some embodiments, scaffolds as provided herein are characterized by morphological features less than 100 nm. Such features have been previously demonstrated for stem cell differentiation (see M. J. Dalby, 6 Nat. Mater., 997-1003 (2007) herein incorporated by reference in its entirety), cell adhesion (see T. J. Webster, 20 Biomaterials, 1221-7 (1999) herein incorporated by reference in its entirety), and metabolic activity (see T. J. Webster, 21 Biomaterials, 1803-10 (2000) herein incorporated by reference in its entirety) and the fabrication of nanostructured scaffolds through nanogelation may be useful to probe biological activity.

In some embodiments, scaffolds are characterized in that when cell-seeded with human fibroblasts, cells showed alignment and increased production of fibrillar nanostructures in the extracellular space.

In some embodiments, scaffolds as provided are capable of seeding and/or functionalization (e.g. with cells and/or other functional moieties). Provided scaffolds are non-toxic and biodegradable. Provided scaffolds are capable of being formed, molded, shaped, and/or machined into desired structures.

In some embodiments, scaffolds of the present disclosure are characterized by particular degradation properties. In some embodiments, scaffolds are degradable.

In some embodiments, scaffolds degrade with a rate that is dependent on a degree of crystallinity within provided scaffolds. In some embodiments, a high degree of crystallinity corresponds with longer biodegradation of the scaffolds. In some embodiments, scaffolds are tunable so that biodegradation of the silk fibroin may be modulated in vivo and in vitro from a period of hours to months to years. While not wishing to be bound to a theory, it is believed that enhanced crystallinity corresponds to a more packed, hydrophobic, structure that decreases accessibility by metalloproteinases (e.g. MMP1, MMP3, MMP9, MMP 13) and other proteolytic enzymes (e.g. chymotrypsin, trypsin) to cleavage sites in the protein (unpublished data).

In some embodiments, scaffolds degrade to release an agent useful for treatment of a disease, disorder, or condition.

In some embodiments, provided silk fibroin-based hydrogel of the present disclosure may be a three-dimensional (3D) structure, wherein at least one dimension of the 3D structure is at least than 10 micrometer.

In some embodiments, provided silk fibroin-based hydrogel may be a 3D structure comprising a predetermined microstructure fabricated therein and/or thereon. In some embodiments, such predetermined microstructure include cavities below its surface.

In some embodiments, scaffolds as described herein are useful for sensor applications, such as biosensor. In some embodiments, sensor agents are included within such materials to form scaffolds.

In some embodiments, materials are encapsulated or infused with dyes, fluorescent materials, or other reactive or sensitive entities. In some embodiments, scaffolds are formed from materials including such agents include cavities arranged and constructed to provide access to such sensor agents.

### Hydrogels

In some embodiments, provided scaffold materials are or are made of hydrogels.

In some embodiments, provided scaffolds are or include at least 50% water. In some embodiments, provided scaffolds are made a hydrogel that includes at least 75% water.

In some embodiments, provided scaffolds include at least 50% water, at least 55% water, at least 60% water, at least 65% water, at least 70% water, at least 75% water, at least 80% water, at least 85% water, at least 90% water, at least 95% water, or more.

In some embodiments, when hydrogel material is removed to form cavities therein; removed material is deposited on an interior surface of such cavities, channels, openings, tunnels, vasculature, or voids. In some embodiments, cavities formed within a hydrogel have an interior surface that is or includes hydrogel material deposited thereon.

In some embodiments, deposited hydrogel material that includes at least 75% water, at least 80% water, at least 85% water, at least 90% water, at least 95% water, or more does not foul.

In some embodiments, materials for use in accordance with present disclosure are polymer hydrogels. In some embodiments, such hydrogels are or include proteins. In some embodiments, hydrogels are or include natural or synthetic proteins. In some embodiments, as provided above, protein polymers of are selected from the group consisting of agarose, alginate, amyloid, cellulose, chitin, chitosan, collagen, elastin, gelatin, hyaluronic acid, pullan, resilin, silk, starch, or combinations thereof.

Among other things, the present disclosure identifies characteristics of silk fibroin hydrogels that render them particularly useful in the context of scaffolds as disclosed or method or technologies of forming scaffolds as provided herein. In some embodiments, the present disclosure utilizes a hydrogel comprised of a polymer that includes or consists of silk fibroin. In some embodiments, a hydrogel includes a polymer that consists of silk fibroin, so that silk fibroin is the only polymer making up the hydrogel, but the hydrogel may have or be formed from one or more other non-polymer components. In some embodiments, a hydrogel includes a protein; in some such embodiments the protein includes or consists of silk fibroin. In some embodiments, such a protein consists of silk fibroin, so that silk fibroin is the only protein making up the hydrogel, but the hydrogel may have or be formed from one or more other non-protein components, including in some embodiments one or more non-protein polymers.

Those of ordinary skill in the art reading the present disclosure will appreciate that a hydrogel that is formed from only a single polymer (or only a single protein) may be seeded or impregnated with one or more other polymers (or proteins). In such a circumstance, the hydrogel is considered to be formed from (i.e., comprised of) only the single polymer (or protein), but the scaffold includes the hydrogel and the seeded/impregnated polymer (or protein).

In some embodiments, scaffolds are or include amino acid phenolic side chains. In some embodiments, a protein polymer solution is a silk fibroin solution. In some embodiments, such as silk, amino acid phenolic side chains are tyrosine.

In some embodiments, hydrogels are manufactured from commonly available reagents. In some embodiments, hydrogels can be produced without needing volatile chemicals to induce gelation. In some embodiments, hydrogels can be produced using all aqueous processing. In some embodiments, hydrogels can be produced without the need to rely on expensive or complicated equipment (power supply, sonicator, vortexer, etc.). In some embodiments, hydrogels are inexpensive to prepare, easy to prepare, and capable of bulk manufacturing.

In some embodiments, hydrogels are prepared under mild, physiologically relevant reaction conditions. In some embodiments, mild aqueous processing is amenable to incorporation of cells and bioactive molecules during formation. In some embodiments, hydrogels provide for ease of infiltration of for example cells, proteins, amino acids, and/or peptides. In some embodiments, the hydrogels are biocompatible and biodegradable. In some embodiments, hydrogels are not cytotoxic. In some embodiments, hydrogels are non-immunogenic.

In some embodiment, hydrogels are formed from silk fibroin solutions. In some embodiments, a hydrogel of the present disclosure is formed from a solution having a protein to solvent concentration between about 0.1 wt % to about 30 wt %. In some embodiments, a hydrogel is formed from a protein polymer having a molecular weight between about 10 kDa and about 400 kDa.

In some embodiments, protein polymers for use in accordance with the present disclosure include amino acid phenolic side chains. In some embodiments, amino acid phenolic side chains are tyrosine.

In some embodiments, scaffolds are formed when polymer chains cross-link into networks through chemical or physical means. In some embodiments, scaffolds of the present disclosure include enzymatically covalently crosslinked phenolic amino acid side chain chains. In some embodiments, enzymatically covalently crosslinked amino acid phenolic side chains are dityrosine covalent bonds. In some embodiments, enzymatically covalently crosslinked phenolic amino acid side chain chains are covalently crosslinked dityrosine bonds.

In some embodiments, provided scaffolds are characterized by their water content. In some embodiments, provided scaffolds include about 30% to about 90% water. In some embodiments, provided scaffolds include about 30% to about 90% water. In some embodiments, provided scaffolds include about 35% to about 90% water. In some embodiments, provided scaffolds include about 40% to about 90% water. In some embodiments, provided scaffolds include about 45% to about 90% water. In some embodiments, provided scaffolds include about 50% to about 90% water. In some embodiments, provided scaffolds include about 55% to about 90% water. In some embodiments, provided scaffolds include about 60% to about 90% water. In some embodiments, provided scaffolds include about 65% to about 90% water. In some embodiments, provided scaffolds include about 70% to about 90% water. In some embodiments, provided scaffolds include about 75% to about 90% water. In some embodiments, provided scaffolds include about 80% to about 90% water. In some embodiments, provided scaffolds include about 85% to about 90% water.

In some embodiments, provided scaffolds include about 30% water. In some embodiments, provided scaffolds include about 35% water. In some embodiments, provided scaffolds include about 40% water. In some embodiments, provided scaffolds include about 45% water. In some embodiments, provided scaffolds include about 50% water. In some embodiments, provided scaffolds include about 55% water. In some embodiments, provided scaffolds include about 60% water. In some embodiments, provided scaffolds include about 65% water. In some embodiments, provided scaffolds include about 70% water. In some embodiments, provided scaffolds include about 71% water. In some embodiments, provided scaffolds include about 72% water. In some embodiments, provided scaffolds include about 73% water. In some embodiments, provided scaffolds include about 74% water. In some embodiments, provided scaffolds include about 75% water. In some embodiments, provided scaffolds include about 76% water. In some embodiments, provided scaffolds include about 77% water. In some embodiments, provided scaffolds include about 78% water. In some embodiments, provided scaffolds include about 79% water. In some embodiments, provided scaffolds include about 80% water. In some embodiments, provided scaffolds include about 81% water. In some embodiments, provided scaffolds include about 82% water. In some embodiments, provided scaffolds include about 83% water. In some embodiments, provided scaffolds include about 84% water. In some embodiments, provided scaffolds include about 85% water. In some embodiments, provided scaffolds include about 86% water. In some embodiments, provided scaffolds include about 87% water. In some embodiments, provided scaffolds include about 88% water. In some embodiments, provided scaffolds include about 89% water. In some embodiments, provided scaffolds include about 90% water. In some embodiments, provided scaffolds include about 91% water. In some embodiments, provided scaffolds include about 92% water. In some embodiments, provided scaffolds include about 93% water. In some embodiments, provided scaffolds include about 94% water. In some embodiments, provided scaffolds include about 95% water. In some embodiments, provided scaffolds include about 96% water or more.

In some embodiments, when water content is greater than about 80%, provided scaffolds are resistant fouling.

In some embodiments, provided scaffolds are characterized by crystalline structure comprising beta sheet structures and/or hydrogen bonding.

In some embodiments, provided scaffolds are configured to support incorporation of at least one agent. In some embodiments, provided scaffolds control release of drugs and other therapeutic agents dispersed therein.

In some embodiments, hydrogels provide ease of incorporation of functional components. In some embodiments, suitable gel functionalization is tunable to specific cell and/or tissue needs. In some embodiments, hydrogels are suitable for functionalization or inclusion of components to support needs of cell engineering or tissue remodeling. In some embodiments, channels molded into scaffolds of materials of the present disclosure support cell infiltration, for example for soft tissue repair and/or replacement by enhancing diffusion of oxygen and nutrients and promoting vascularization in critically sized defects.

In some embodiments, hydrogels are biodegradable. In some embodiments biodegradable hydrogels may be safely incorporated in vivo. In some embodiments, hydrogels degrade to release of agents incorporated therein. In some embodiments, hydrogels are biocompatible and biodegradable. In some embodiments, controlled release of an agent from hydrogels may be designed to occur over time, for example, over 12 hours or 24 hours. The time of release may be selected, for example, to occur over a time period of about 12 hours to 24 hours; about 12 hours to 42 hours; or, e.g., about 12 to 72 hours. In another embodiment, release may occur for example on the order of about 1 day to 15 days. The controlled release time may be selected based on the condition treated. For example, longer times may be more effective for wound healing, whereas shorter delivery times may be more useful for some cardiovascular applications. In some embodiments controlled release of an agent from hydrogels in vivo may occur, for example, in the amount of about 1 ng to 1 mg/day. In other embodiments, the controlled release may occur in the amount of about 50 ng to 500 ng/day, or, in another embodiment, in the amount of about 100 ng/day. Delivery systems comprising therapeutic agent and a carrier may be formulated that include, for example, 10 ng to 1 mg therapeutic agent, or about 1 μg to 500 μm, or, for example, about 10 μg to 100 μg, depending on the therapeutic application. Moreover, in some embodiments, incorporation of growth factors, and/or incorporation other cell signaling factors provide optimization of cell functions.

In some embodiments, provided scaffolds of the present disclosure are characterized by high stiffness and superior resilience and elasticity. In some embodiments, provided scaffolds of the present disclosure are characterized in that they fully recover from large strains or long term cyclic compressions. In some embodiments, provided scaffolds of the present disclosure are characterized in that they withstand long term stress with negligible changes in modulus and without showing an indication of appreciable changes in mechanical properties, such as a plastic deformation. In some embodiments, provided scaffolds of the present disclosure are characterized in that they are capable of withstanding repeated strains. In some embodiments, provided scaffolds of the present disclosure that have been shown to exhibit the above identified characteristics and/or properties are formed from solutions of low weight percent concentration of polymer and of low molecular weight polymers.

In some embodiments, scaffolds made of hydrogels exhibit tunable mechanical properties. In some embodiments, mechanical properties of hydrogels are tunable for use in different applications. In some embodiments, hydrogels may be tailored by tunable properties to specific needs, for example, cell engineering or tissue remodeling. In some embodiments, hydrogels include a polymer having enzymatically covalently crosslinked amino acid phenolic side chains. In some embodiments, hydrogels are characterized by a storage modulus value between about 50 Pa and about 100 kPa without an indication of a plastic deformation. In some embodiments, hydrogels are capable of recovering from a shear strain of at least 100% without showing an indication of a plastic deformation. In some embodiments, hydrogels provide a tangent modulus between about 200 Pa to about 400 kPa. In some embodiments, hydrogels are capable of recovering from a compressive strain of at least 75% without showing an indication of a plastic deformation.

In some embodiments, hydrogels swell up to 400% when exposed to solvents. In some embodiments, hydrogels are configured to support cell encapsulation. In some embodiments, hydrogels provide direct encapsulation of cells. In some embodiments, encapsulated cells show long term survival. In some embodiments, hydrogels support cell survival and proliferation and were well tolerated when implanted in vivo. In some embodiments, hydrogels form a matrix for supporting cell encapsulation.

In some embodiments, hydrogels provide control of cell-matrix interactions. In some embodiments, control of cell-matrix interactions is reflective of varying protein polymer solution concentration and/or modifying gelation conditions. In some embodiments, control of cell matrix interactions influence cell shape. In some embodiments, influencing cell shapes provides for differentiation of cells. In some embodiments, hydrogels are biodegradable. In some embodiments, biodegradable hydrogels provide controlled delivery of agents. In some embodiments, biomaterials and methods described herein include or include biologically degradable hydrogels that carry agents to induce repair and/or remodeling of tissues and cells.

In some embodiments, hydrogels are useful for in vivo and in vitro applications requiring highly resilient, tunable, elastomeric substrates. In some embodiments, tunable elastic materials a usable in injectable systems. In some embodiments, hydrogels support cell engineering and/or tissue regeneration thereby preventing a disease, disorder or condition and/or inducing repair site.

In some embodiments, enzymatic crosslinking provides in the formation of optically clear gels with negligible absorbance above 350 nm. In some embodiments, the hydrogels provide a platform for incorporation of optical or optoelectronic devices. In some embodiments, hydrogels are optically clear. In some embodiments, hydrogels are configured to be optical materials.

Silks

In some embodiments, a scaffold is or includes silk, or one or more silk proteins (e.g., silk fibroin as discussed below).

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers.

Silk has been a highly desired and widely used textile since its first appearance in ancient China. (See Elisseeff, “The Silk Roads: Highways of Culture and Commerce,” Berghahn Books/UNESCO, New York (2000); see also Vainker, “Chinese Silk: A Cultural History,” Rutgers University Press, Piscataway, N.J. (2004)). Glossy and smooth, silk is favored by not only fashion designers but also tissue engineers because it is mechanically tough but degrades harmlessly inside the body, offering new opportunities as a highly robust and scaffold substrate. (See Altman et al., Biomaterials, 24: 401 (2003); see also Sashina et al., Russ. J. Appl. Chem., 79: 869 (2006)).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

In general, silk for use in accordance with the present disclosure may be produced by any such organism, or may be prepared through an artificial process, for example, involving genetic engineering of cells or organisms to produce a silk protein and/or chemical synthesis. In some embodiments of the present disclosure, silk is produced by the silkworm, Bombyx mori.

As is known in the art, silks are modular in design, with large internal repeats flanked by shorter (˜100 amino acid) terminal domains (N and C termini). Naturally-occurring silks have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules. Table 1, below, provides an exemplary list of silk-producing species and silk proteins:

TABLE 1
An exemplary list of silk-producing species and silk proteins
(adopted from Bini et al. 335 J. Mol. Biol. 1, 27-40 (2003)).

| | | | |
|---|---|---|---|
| Accession | Species | Producing gland | Protein |

A. Silkworms

| | | | |
|---|---|---|---|
| AAN28165 | Antheraea mylitta | Salivary | Fibroin |
| AAC32606 | Antheraea pernyi | Salivary | Fibroin |
| AAK83145 | Antheraea yamamai | Salivary | Fibroin |
| AAG10393 | Galleria mellonella | Salivary | Heavy-chain |
| | | | fibroin |
| | | | (N-terminal) |
| AAG10394 | Galleria mellonella | Salivary | Heavy-chain |
| | | | fibroin |
| | | | (C-terminal) |
| P05790 | Bombyx mori | Salivary | Fibroin heavy |
| | | | chain |
| | | | precursor, |
| | | | Fib-H, H- |
| | | | fibroin |
| CAA27612 | Bombyx mandarina | Salivary | Fibroin |
| Q26427 | Galleria mellonella | Salivary | Fibroin light |
| | | | chain precur- |
| | | | sor, Fib-L, |
| | | | L-fibroin, |
| | | | PG-1 |
| P21828 | Bombyx mori | Salivary | Fibroin light |
| | | | chain precur- |
| | | | sor, Fib-L, |
| | | | L-fibroin |

B. Spiders

| | | | |
|---|---|---|---|
| P19837 | Nephila clavipes | Major ampullate | Spidroin 1, |
| | | | dragline silk |
| | | | fibroin 1 |
| P46804 | Nephila clavipes | Major ampullate | Spidroin 2, |
| | | | dragline silk |
| | | | fibroin 2 |
| AAK30609 | Nephila senegalensis | Major ampullate | Spidroin 2 |
| AAK30601 | Gasteracantha | Major ampullate | Spidroin 2 |
| | mammosa | | |
| AAK30592 | Argiope aurantia | Major ampullate | Spidroin 2 |
| AAC47011 | Araneus diadematus | Major ampullate | Fibroin-4, |
| | | | ADF-4 |
| AAK30604 | Latrodectus | Major ampullate | Spidroin 2 |
| | geometricus | | |
| AAC04503 | Araneus bicentenarius | Major ampullate | Spidroin 2 |
| AAK30615 | Tetragnatha versicolor | Major ampullate | Spidroin 1 |
| AAN85280 | Araneus ventricosus | Major ampullate | Dragline silk |
| | | | protein-1 |
| AAN85281 | Araneus ventricosus | Major ampullate | Dragline silk |
| | | | protein-2 |
| AAC14589 | Nephila clavipes | Minor ampullate | MiSp1 silk |
| | | | protein |
| AAK30598 | Dolomedes tenebrosus | Ampullate | Fibroin 1 |
| AAK30599 | Dolomedes tenebrosus | Ampullate | Fibroin 2 |
| AAK30600 | Euagrus chisoseus | Combined | Fibroin 1 |
| AAK30610 | Plectreurys tristis | Larger ampule- | Fibroin 1 |
| | | shaped | |
| AAK30611 | Plectreurys tristis | Larger ampule- | Fibroin 2 |
| | | shaped | |
| AAK30612 | Plectreurys tristis | Larger ampule- | Fibroin 3 |
| | | shaped | |
| AAK30613 | Plectreurys tristis | Larger ampule- | Fibroin 4 |
| | | shaped | |
| AAK30593 | Argiope trifasciata | Flagelliform | Silk protein |
| AAF36091 | Nephila | Flagelliform | Fibroin, silk |
| | madagascariensis | | protein |
| | | | (N-terminal) |
| AAF36092 | Nephila | Flagelliform | Silk protein |
| | madagascariensis | | (C-terminal) |
| AAC38846 | Nephila clavipes | Flagelliform | Fibroin, silk |
| | | | protein |
| | | | (N-terminal) |
| AAC38847 | Nephila clavipes | Flagelliform | Silk protein |
| | | | (C-terminal) |

Silk Fibroin

Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 kDa) and the fibroin light chain (˜25 kDa), which are associated with a family of non-structural proteins termed sericin, which glue the fibroin brings together in forming the cocoon. The heavy and light chains of fibroin are linked by a disulfide bond at the C-terminus of the two subunits. (See Takei, F., et al., 105 J. Cell Biol., 175-180 (1987); see also Tanaka, K., et al., 114 J. Biochem. (Tokyo), 1-4 (1993); Tanaka, K., et al., 1432 Biochim. Biophys. Acta., 92-103 (1999); Y Kikuchi, et al., “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

As used herein, the term “silk fibroin” refers to silk fibroin protein, whether produced by silkworm, spider, or other insect, or otherwise generated. (See Lucas et al., 13 Adv. Protein Chem., 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

Thus, in some embodiments, a silk solution is used to fabricate compositions of the present disclosure contain fibroin proteins, essentially free of sericins. In some embodiments, silk solutions used to fabricate various compositions of the present disclosure contain the heavy chain of fibroin, but are essentially free of other proteins. In other embodiments, silk solutions used to fabricate various compositions of the present disclosure contain both the heavy and light chains of fibroin, but are essentially free of other proteins. In certain embodiments, silk solutions used to fabricate various compositions of the present disclosure include both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

Silk materials explicitly exemplified herein were typically prepared from material spun by silkworm, Bombyx mori. Typically, cocoons are boiled in an aqueous solution of 0.02 M Na₂CO₃, then rinsed thoroughly with water to extract the glue-like sericin proteins. Extracted silk is then dissolved in a solvent, for example, LiBr (such as 9.3 M) solution at room temperature. A resulting silk fibroin solution can then be further processed for a variety of applications as described elsewhere herein.

In some embodiments, polymers refers to peptide chains or polypeptides having an amino acid sequence corresponding to fragments derived from silk fibroin protein or variants thereof.

In the context of hydrogels, silk fibroin fragments generally refer to silk fibroin peptide chains or polypeptides that are smaller than naturally occurring full length silk fibroin counterpart, such that one or more of the silk fibroin fragments within a population or composition. In some embodiments, for example, scaffolds include silk fibroin polypeptides having an average molecular weight of between about 3.5 kDa and about 350 kDa. In some embodiments, suitable ranges of silk fibroin fragments include, but are not limited to: silk fibroin polypeptides having an average molecular weight of between about 3.5 kDa and about 200 kDa; silk fibroin polypeptides having an average molecular weight of between about 3.5 kDa and about 150 kDa; silk fibroin polypeptides having an average molecular weight of between about 3.5 kDa and about 120 kDa. In some embodiments, silk fibroin polypeptides have an average molecular weight of: about 3.5 kDa, about 4 kDa, about 4.5 kDa, about 5 kDa, about 6 kDa, about 7 kDa, about 8 kDa, about 9 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 150 kDa, about 200 kDa, about 250 kDa, about 300 kDa, or about 350 kDa. In some preferred embodiments, silk fibroin polypeptides have an average molecular weight of about 100 kDa.

In some embodiments, scaffolds are or include silk fibroin, fibroin fragments, and/or silk fibroin hydrogels. In some embodiments, silk fibroin and/or silk fibroin fragments of various molecular weights may be used. In some embodiments, silk fibroin and/or silk fibroin fragments of various molecular weights are silk fibroin polypeptides. In some embodiments, silk fibroin polypeptides are “reduced” in size, for instance, smaller than the original or wild type counterpart, may be referred to as “low molecular weight silk fibroin.” For more details related to low molecular weight silk fibroins, see: U.S. provisional application concurrently filed herewith, entitled “LOW MOLECULAR WEIGHT SILK FIBROIN AND USES THEREOF,” the entire contents of which are incorporated herein by reference. In some embodiments, silk fibroin polypeptides have an average molecular weight of: less than 350 kDa, less than 300 kDa, less than 250 kDa, less than 200 kDa, less than 175 kDa, less than 150 kDa, less than 120 kDa, less than 100 kDa, less than 90 kDa, less than 80 kDa, less than 70 kDa, less than 60 kDa, less than 50 kDa, less than 40 kDa, less than 30 kDa, less than 25 kDa, less than 20 kDa, less than 15 kDa, less than 12 kDa, less than 10 kDa, less than 9 kDa, less than 8 kDa, less than 7 kDa, less than 6 kDa, less than 5 kDa, less than 4 kDa, less than 3.5 kDa, less than 3 kDa, less than 2.5 kDa, less than 2 kDa, less than 1.5 kDa, or less than about 1.0 kDa, etc.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, hydrogels produced from silk fibroin fragments can be formed by degumming silk cocoons in an aqueous solution at temperatures of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C.

In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, silk fibroin fragments solubilized prior to gelation. In some embodiments, a carrier can be a solvent or dispersing medium. In some embodiments, a solvent and/or dispersing medium, for example, is water, cell culture medium, buffers (e.g., phosphate buffered saline), a buffered solution (e.g. PBS), polyol (for example, glycerol, propylene glycol, liquid polyethylene glycol, and the like), Dulbecco's Modified Eagle Medium, fetal bovine serum, or suitable combinations and/or mixtures thereof.

In some embodiments, scaffolds are modulated by controlling a silk concentration. In some embodiments, a weight percentage of silk fibroin can be present in the solution at any concentration suited to the need. In some embodiments, an aqueous silk fibroin solution can have silk fibroin at a concentration of about 0.1 mg/mL to about 20 mg/mL. In some embodiments, an aqueous silk fibroin solution can include silk fibroin at a concentration of about less than 1 mg/mL, about less than 1.5 mg/mL, about less than 2 mg/mL, about less than 2.5 mg/mL, about less than 3 mg/mL, about less than 3.5 mg/mL, about less than 4 mg/mL, about less than 4.5 mg/mL, about less than 5 mg/mL, about less than 5.5 mg/mL, about less than 6 mg/mL, about less than 6.5 mg/mL, about less than 7 mg/mL, about less than 7.5 mg/mL, about less than 8 mg/mL, about less than 8.5 mg/mL, about less than 9 mg/mL, about less than 9.5 mg/mL, about less than 10 mg/mL, about less than 11 mg/mL, about less than 12 mg/mL, about less than 13 mg/mL, about less than 14 mg/mL, about less than 15 mg/mL, about less than 16 mg/mL, about less than 17 mg/mL, about less than 18 mg/mL, about less than 19 mg/mL, or about less than 20 mg/mL.

In some embodiments, a hydrogel is configured to be injectable. In some embodiments, a viscosity of an injectable composition is modified by using a pharmaceutically acceptable thickening agent. In some embodiments, a thickening agent, for example, is methylcellulose, xanthan gum, carboxymethyl cellulose, hydroxypropyl cellulose, carbomer, or combination thereof. A preferred concentration of the thickener depends upon a selected agent and viscosity for injection.

In some embodiments, hydrogel form a porous matrix or scaffold. For example, the porous scaffold can have a porosity of at least about 10%, at least about 20%, at least about 30%, at least about 40%, at least about 50%, at least about 60%, at least about 70%, at least about 80%, at least about 90%, or higher.

Mechanical Properties of Hydrogels

Excessive local stress and strain have been implicated in pathological remodeling of tissues. In some embodiments, modulus and mechanical stimulation are critical for proper development of cells and/or tissues. In some embodiments, hydrogels are capable of withstanding long term strains without appreciable changes in mechanical properties or plastic deformation.

In some embodiments, scaffolds of the present disclosure and methods of forming scaffolds of the present disclosure provide matching mechanical properties for cells and/or tissues to a native tissue extracellular matrix (ECM). In some embodiments, hydrogels can be fine-tuned to optimize matching to native tissue. In particular, in some embodiments, mechanical properties such as for example, stiffness, elasticity, and or swelling of hydrogels may be fine-tuned to optimize matching.

In some embodiments, hydrogels are tunable to influence cell size and shape. In some embodiments, cell-matrix interactions are influenced by for examples, protein polymer concentration, protein polymer molecular weight and/or gelation conditions.

In some embodiments, hydrogels provide a three dimensional cell culture or matrix.

In some embodiments, network strength of the matrix is a function of elasticity. In some embodiments, tunable properties of hydrogels influence network strength of a matrix. In some embodiments, tunable mechanics provided by hydrogels allow for control of cell-matrix interactions.

In some embodiments, a three dimensional cell culture or matrix supports and may influence cell-specific morphologies, or different morphologies.

In some embodiments, hydrogels influencing cell size and shape, for example stem cells, providing cell differentiation and maintenance of cell phenotype during cell culture.

In some embodiments, hydrogels are tunable to influence cell size and shape. In some embodiments, cell-matrix interactions are influenced by for examples, protein polymer concentration, protein polymer molecular weight and/or gelation conditions. In some embodiments, tunable properties of covalently crosslinked hydrogels displaying stronger network strength and thereby influencing cell shape. In some embodiments, manipulating cell shape provides differentiation of cell type.

In some embodiments, a hydrogel of the present disclosure for use in encapsulating cells includes tunable resilience and/or elasticity between a hydrogel and a native tissue. In some embodiments, mechanical properties provide an indication of resilience and/or elasticity between a hydrogel and a native tissue. In some embodiments, mechanical properties of hydrogels are capable of being matched, tuned, adjusted, and/or manipulated with mechanical properties of cells of hydrogels. In some embodiments, mechanical properties include, for example, storage modulus, tangent modulus, plateau modulus, swelling, and/or dynamic modulus.

In some embodiments, matching, tuning, adjusting, and/or manipulating mechanical properties of a hydrogel include, for example: selecting a molecular weight of a polymer, selecting a concentration of a polymer solution, selecting a specific polymer, selecting a specific peroxidase, selecting a specific peroxide, selecting a concentration of peroxidase, selecting a concentration of peroxide, or combinations thereof.

In some embodiments, hydrogels provide tunable mechanical properties yielding a combination elasticity, resiliency, tunable biodegradability, and cell encapsulation features. In some embodiments, mechanical properties of hydrogels include storage modulus, tangent modulus, plateau modulus, dynamic modulus, and capacity to swell. In some embodiments, hydrogels are characterized by a storage modulus value between about 50 Pa and about 100 kPa without an indication of a plastic deformation. In some embodiments, hydrogels are capable of recovering from a shear strain of at least 100% without showing an indication of a plastic deformation. In some embodiments, hydrogels provide a tangent modulus between about 200 Pa to about 400 kPa. In some embodiments, hydrogels are capable of recovering from a compressive strain of at least 75% without showing an indication of a plastic deformation.

In some embodiments, hydrogels fully recover from large strains and/or long term cyclic compressions. In some embodiments, hydrogels were exposed to large strains, up to 80% strain. In some embodiments, hydrogels were exposed to long term cyclic compressions, 10% strain at a frequency of 0.5 Hz and 3,600 cycles.

In some embodiments, hydrogels provide tunable mechanical properties by selecting a polymer solution concentration between about 0.1 wt % and about 30 wt %.

Indeed in some embodiments, provided scaffolds formed from low weight percent polymer solutions, that is below 10 wt %, surprisingly have exhibited desired properties and/or characteristics in the above mentioned ranges, specifically desired high storage modulus values that prior gels were not able to achieve. The present disclosure thus identifies the source of a problem with certain other hydrogel technologies in that they typically require a higher weight percent of polymer in solution to achieve desired properties, such as high storage moduli.

In some embodiments, hydrogels provide tunable mechanical properties by selecting a molecular weight of a polymer, such as silk fibroin between about 10 kDa and about 400 kDa. In some embodiments, molecular weight is variable by adjusting boiling time. In some embodiments, molecular weight is inversely proportional to a boiling time.

In some embodiments, hydrogels provide tunable mechanical properties by changing a solvent present in a polymer solution, such a silk fibroin solution. In some embodiments, hydrogels provide tunable mechanical properties by changing a type of amino acid incorporated on the polymer. In some embodiments, hydrogels provide tunable mechanical properties by changing a type of peroxidase. In some embodiments, hydrogels provide tunable mechanical properties by changing a peroxidase concentration. In some embodiments, hydrogels provide tunable mechanical properties by changing a type of peroxide. In some embodiments, hydrogels provide tunable mechanical properties by changing a peroxide concentration.

In some embodiments, three dimensional cell culture supports are further tunable so that cell-matrix interactions in vivo may be exploited to control integration of the biomaterials following implantation. In some embodiments, suitable hydrogel functionalization supports for examples inclusion of ECM components, incorporation of growth factors and incorporation of other cell signaling factors to optimize cell functions. In some embodiments, molding of channels into scaffolds was advantageous for cell infiltration for soft tissue repair and replacement. In some embodiments, a linear wire array forms channels throughout a scaffold to enhance diffusion of oxygen and nutrients and promote vascularization in critically sized defects. In some embodiments, molding of channels into scaffolds and allowing for implantation of larger scaffolds without concern for necrosis due to diffusion limits. In some embodiments, influencing cell-matrix interactions provides control of a hydrogel degradation rate.

In some embodiments, matching, tuning, adjusting, and/or manipulating mechanical properties of a hydrogel of the present disclosure for use in encapsulating cells and/or influencing cell shape is accomplished by selecting a molecular weight of a polymer. In some embodiments, a molecular weight of a polymer is in a range of molecular weights between about 10 kDa and about 400 kDa.

In some embodiments, matching, tuning, adjusting, and/or manipulating mechanical properties of a hydrogel of the present disclosure for use in encapsulating cells and/or influencing cell shape includes is accomplished by selecting a polymer solution concentration. In some embodiments, a polymer solution concentration is in a range of concentrations between about 0.1 wt % and about 30 wt %.

In some embodiments, scaffolds of the present disclosure may have pores therein, i.e., a measurable degree of porosity. For example, in some embodiments, provided scaffolds have a porosity of between about 0% and 50%, e.g., about 5%, about 10%, about 15%, about 20%, about 25%, about 30%, about 35%, about 40%, about 45%, about 50%, etc. Suitable porogens, for example, may be used to achieve desired porosity.

In some embodiments, matching, tuning, adjusting, and/or manipulating mechanical properties of a hydrogel of the present disclosure for use in encapsulating cells and/or influencing cell shape includes is accomplished by selecting a molecular weight of a polymer and selecting a polymer solution concentration.

In some embodiments, a method of providing, preparing, and/or manufacturing a hydrogel of the present disclosure includes controlling a rate of degradation of a hydrogel of the present disclosure for maintaining a hydrogel shape, optimizing infiltration and/or integration of a covalently crosslinked hydrogel, maximizing cell spreading, and releasing a prescribed amount of an agent or a moiety from a hydrogel over a time. In some embodiments, a rate of degradation of a hydrogel may be controlled by selecting a molecular weight of a polymer, by selecting a polymer solution concentration, by selecting a specific polymer, by selecting a specific peroxidase, by selecting a specific peroxide, and combinations thereof.

In some embodiments, infusing oxygen and nutrients into a matrix encapsulating cells enhances cell infiltration and soft tissue repair. In some embodiments, a method manufacturing a hydrogel of the present disclosure includes molding channels into a hydrogel matrix encapsulating cells to enhance diffusion of oxygen and nutrients and promote cell vascularization.

Tunable Mechanical Properties

In some embodiments, provided scaffolds are characterized such that in that have widely tunable mechanical properties. In some embodiments, provided scaffolds that exhibit tunable mechanical properties provide flexibility in downstream applications.

In some embodiments, scaffolds of the present disclosure are characterized by highly tunable mechanical properties. In some embodiments, scaffolds of the present disclosure are characterized in that they possess mechanical properties that are tunable to a particular desired range and/or set. In some embodiments, mechanical properties, in particular compressive strength and compressive modulus are tunable.

In some embodiments, a compressive strength of scaffolds is tunable in a range of between about 0.5 kPa and about 12 kPa without showing an indication of a plastic deformation. In some embodiments, scaffolds show a compressive strength of about 0.5 kPa, about 1 kPa, about 1.5 kPa, about 2 kPa, about 2.5 kPa, about 3 kPa, about 3.5 kPa, about 4 kPa, about 4.5 kPa, about 5 kPa, about 5.5 kPa, about 6 kPa, about 7 kPa, about 8 kPa, about 9 kPa, about 10 kPa, about 11 kPa, or about 12 kPa without showing an indication of a plastic deformation.

In some embodiments, a compressive modulus of scaffolds is tunable in a range of between about 0.5 kPa and about 20 kPa without showing an indication of a plastic deformation when measure at crosshead rates of: 0.100 mm/min, 0.200 mm/min, and/or 2.00 mm/min. In some embodiments, scaffolds show a compressive modulus of about 0.5 kPa, about 1 kPa, about 2 kPa, about 3 kPa, about 4 kPa, about 5 kPa, about 6 kPa, about 7 kPa, about 8 kPa, about 9 kPa, about 10 kPa, about 11 kPa, about 12 kPa, about 13 kPa, about 14 kPa, about 15 kPa, about 16 kPa, about 17 kPa, about 18 kPa, about 19 kPa or about 20 kPa without showing an indication of a plastic deformation when measure at crosshead rates of: 0.100 mm/min, 0.200 mm/min, or 2.00 mm/min.

In some embodiments, at least part of such elasticity or compressive ability may be facilitated by crosslinking agent. In some embodiments, a crosslinking agent is or includes EDTA, or agent having a similar activity. In some embodiments, between about 1 mM and 100 mM EDTA may be used to carry out a crosslinking step. In some embodiments, about 5 mM, about 10 mM, about 15 mM, about 20 mM, about 30 mM, about 40 mM, or about 50 mM EDTA may be used to carry out crosslinking. In some embodiments, such crosslinking step may be carried out for about a few seconds, minutes, to hours.

In some embodiments, such a crosslinking step may be carried out by exposing a silk fibroin-based hydrogel as provided herein with a crosslinking agent, such as EDTA for about 0.5 hour, about 1.0 hour, about 1.5 hours, about 2.0 hours, about 2.5 hours, about 3.0 hours, about 3.5 hours, about 4.0 hours, about 4.5 hours, about 5.0 hours, about 5.5 hours, about 6.0 hours, about 6.5 hours, about 7.0 hours, about 7.5 hours, about 8.0 hours, about 8.5 hours, about 9.0 hours, about 9.5 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or longer.

In some embodiments, scaffolds with tunable properties are characterized in that they exhibit improved structural stability corresponding to increased compressive strength and/or increased compressive modulus. In some embodiments, at least part of such elasticity or compressive ability may be facilitated by crosslinking agent. In some embodiments, for example, one or more crosslinking agents may be used to achieve crosslinking of silk fibroin polypeptides, intra-molecularly, inter-molecularly, or both. Any suitable crosslinking agents may be used, including but are not limited to: an amine-to-amine crosslinker, amine-to-sulfhydryl crosslinker, carboxyl-to-amine crosslinker, photoreactive crosslinker, sulfhydryl-to-carbohydrate crosslinker, sulfhydryl-to-hydroxyl crosslinker, sulfhydryl-to-sulfhydryl crosslinker, or any combination thereof.

Transparency

In some embodiments, scaffolds exhibit transparency in visual spectrum. In some embodiments, scaffolds transmit at least some light in a wavelength range between about 200 nm to about 1750 nm. In some embodiments, provided scaffolds are between about 50% and 100% transparent in the visible spectrum. In some embodiments, scaffolds are characterized by having a high degree of transparency, e.g., about 20% to 99% transmittance for wavelengths ranging between about 200 nm to about 1750 nm.

In some embodiments, scaffolds of the present disclosure are characterized by optical transmittance greater than 90% for wavelengths ranging between about 200 nm to about 1750 nm. In some embodiments, scaffolds of the present disclosure are characterized by optical transmittance greater than 91% for wavelengths ranging between about 200 nm to about 1750 nm. In some embodiments, scaffolds of the present disclosure are characterized by optical transmittance greater than 92% for wavelengths ranging between about 200 nm to about 1750 nm. In some embodiments, scaffolds of the present disclosure are characterized by optical transmittance greater than 93% for wavelengths ranging between about 200 nm to about 1750 nm. In some embodiments, scaffolds of the present disclosure are characterized by optical transmittance greater than 94% for wavelengths ranging between about 200 nm to about 1750 nm. In some embodiments, scaffolds of the present disclosure are characterized by optical transmittance greater than 95% for wavelengths ranging between about 200 nm to about 1750 nm. In some embodiments, scaffolds of the present disclosure are characterized by optical transmittance greater than 96% for wavelengths ranging between about 200 nm to about 1750 nm. In some embodiments, scaffolds of the present disclosure are characterized by optical transmittance greater than 97% for wavelengths ranging between about 200 nm to about 1750 nm. In some embodiments, scaffolds of the present disclosure are characterized by optical transmittance greater than 98% for wavelengths ranging between about 200 nm to about 1750 nm. In some embodiments, scaffolds of the present disclosure are characterized by optical transmittance greater than 99% for wavelengths ranging between about 200 nm to about 1750 nm.

In some embodiments, scaffolds are at least 35% transparent in the visible spectrum, at least 40% transparent in the visible spectrum, at least 45% transparent in the visible spectrum, at least 50% transparent in the visible spectrum, at least 55% transparent in the visible spectrum, at least 60% transparent in the visible spectrum, at least 65% transparent in the visible spectrum, at least 70% transparent in the visible spectrum, at least 75% transparent in the visible spectrum, at least 80% transparent in the visible spectrum, at least 85% transparent in the visible spectrum, at least 90% transparent in the visible spectrum, at least 91% transparent in the visible spectrum, at least 92% transparent in the visible spectrum, at least 93% transparent in the visible spectrum, at least 94% transparent in the visible spectrum, at least 95% transparent in the visible spectrum at least 96% transparent in the visible spectrum, at least 97% transparent in the visible spectrum, at least 98% transparent in the visible spectrum, at least 99% transparent in the visible spectrum, or greater transparency in the visible spectrum as determined by methods described herein

In some embodiments, scaffolds are characterized as exhibiting crystallinity, nanosized crystalline particles, and optical transparency as described hereinabove at least part of which is facilitated by a sol-gel transition of a silk fibroin solution to a silk fibroin-based hydrogel via a nanogelation as provided herein.

In some embodiments, nanogelation of a silk fibroin solution to form scaffolds as provided herein occurs that mixing a silk fibroin solution with a polar organic solvent. In some embodiments, a polar organic solvent is or includes acetone, ethanol, methanol, isopropanol, or combinations thereof. In some preferred embodiments, a polar organic solvent is acetone. In some embodiments, a silk fibroin solution has a concentration of about 0.1 mg/mL to about 20 mg/mL. In some preferred embodiments, a silk fibroin solution has a concentration of less than about 15 mg/mL. In some preferred embodiments, a silk fibroin solution has a concentration of less than about 10 mg/mL. In some embodiments, polar organic solvents drives the assembly of silk micelles into submicron-sized particles (<100 nm) to form scaffolds characterized as having optical transparency.

In some embodiments, scaffolds exhibit increased transparency when compared with traditional hydrogels, such as collagen based hydrogels.

Degradation Properties of Silk-Based Materials

Additionally, as will be appreciated by those of skill in the art, much work has established that researchers have the ability to control the degradation process of silk. According to the present disclosure, such control can be particularly valuable in the fabrication of electronic components, and particularly of electronic components that are themselves and/or are compatible with biomaterials. Degradability (e.g., bio-degradability) is often essential for biomaterials used in tissue engineering and implantation. The present disclosure encompasses the recognition that such degradability is also relevant to and useful in the fabrication of silk electronic components.

According to the present disclosure, one particularly desirable feature of silk-based materials is the fact that they can be programmably degradable. That is, as is known in the art, depending on how a particular silk-based material is prepared, it can be controlled to degrade at certain rates. Degradability and controlled release of a substance from silk-based materials have been published (see, for example, WO 2004/080346, WO 2005/012606, WO 2005/123114, WO 2007/016524, WO 2008/150861, WO 2008/118133, each of which is incorporated by reference herein).

Control of silk material production methods as well as various forms of silk-based materials can generate silk compositions with known degradation properties. For example, using various silk fibroin materials (e.g., microspheres of approximately 2 μm in diameter, silk film, silk hydrogels) entrapped agents such as therapeutics can be loaded in active form, which is then released in a controlled fashion, e.g., over the course of minutes, hours, days, weeks to months. It has been shown that layered silk fibroin coatings can be used to coat substrates of any material, shape and size, which then can be used to entrap molecules for controlled release, e.g., 2-90 days.

Crystalline Silk Materials

As known in the art and as described herein, silk proteins can stack with one another in crystalline arrays. Various properties of such arrays are determined, for example, by the degree of beta-sheet structure in the material, the degree of cross-linking between such beta sheets, the presence (or absence) of certain dopants or other materials. In some embodiments, one or more of these features is intentionally controlled or engineered to achieve particular characteristics of a silk matrix. In some embodiments, scaffolds are characterized by crystalline structure, for example, comprising beta sheet structure and/or hydrogen bonding. In some embodiments, provided scaffolds are characterized by a percent beta sheet structure within the range of about 0% to about 45%. In some embodiments, scaffolds are characterized by crystalline structure, for example, comprising beta sheet structure of about 1%, about 2%, about 3%, about 4%, about 5%, about 6%, about 7%, about 8%, about 9%, about 10%, about 11%, about 12%, about 13%, about 1%, about 1%, about 1%, about 1%, about 1%, about 1%, about 1%, about 1%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45%.

Nanosized Crystalline Particles

In some embodiments, scaffolds are characterized in that they include submicron size or nanosized crystallized spheres and/or particles. In some embodiments, such submicron size or nanosized crystallized spheres and/or particles have average diameters ranging between about 5 nm and 200 nm. In some embodiments, submicron size or nanosized crystallized spheres and/or particles have less than 150 nm average diameter, e.g., less than 145 nm, less than 140 nm, less than 135 nm, less than 130 nm, less than 125 nm, less than 120 nm, less than 115 nm, less than 110 nm, less than 100 nm, less than 90 nm, less than 80 nm, less than 70 nm, less than 60 nm, less than 50 nm, less than 40 nm, less than 30 nm, less than 20 nm, less than 15 nm, less than 10 nm, less than 5 nm, or smaller. In some preferred embodiments, submicron size or nanosized crystallized spheres and/or particles have average diameters of less than 100 nm.

### Functionalized Scaffolds

In some embodiments, scaffolds include additives, agents, and/or functional moieties. In some embodiments, provided scaffolds are characterized in that they are capable of supporting biological activity. In some embodiments, scaffolds include biologically active additives, agents, or functional moieties. In some embodiments, when biologically active additives, agents, or functional moieties are incorporated with or present in provided scaffolds, they retain their biological properties and activity. In some embodiments, when biologically active additives, agents, or moieties are incorporated with or present in provided scaffolds, their biological properties and activity are not reduced or inhibited.

In some embodiments, cells, tissues, organs, or sensors applications rely on active biological materials. In some embodiments, additives, agents and/or functional moieties may be added to scaffold to support cell growth.

In some embodiments, additives, agents and/or functional moieties are uniformly dispersed within a scaffold. In some embodiments, additives, agents and/or functional moieties are uniformly dispersed on a surface of a scaffold. In some embodiments, additives, agents and/or functional moieties are dispersed within a scaffold according to a gradient. In some embodiments, a gradient varies with density or concentration, for example, from areas of high density or concentration to areas of low density or concentration. In some embodiments, additives, agents and/or functional moieties are randomly dispersed within a scaffold. In some embodiments, additives, agents and/or functional moieties are uniformly dispersed on a surface of a scaffold. In some embodiments, additives, agents and/or functional moieties are dispersed on a surface of a scaffold according to a gradient. In some embodiments, additives, agents and/or functional moieties are randomly dispersed on a surface of a scaffold.

In some embodiments, provided scaffolds can include one or more (e.g., one, two, three, four, five or more) agents and/or functional moieties (together, “additives”). Without wishing to be bound by a theory additive can provide or enhance one or more desirable properties, e.g., strength, flexibility, ease of processing and handling, biocompatibility, bioresorability, surface morphology, release rates and/or kinetics of one or more active agents present in the composition, and the like. In some embodiments, one or more such additives can be covalently or non-covalently linked with the hydrogel (e.g., with a polymer such as silk fibroin that makes up the hydrogel) and can be integrated homogenously or heterogeneously within the silk composition.

In some embodiments, an additive is or includes a moiety covalently associated (e.g., via chemical modification or genetic engineering) with a polymer. In some embodiments, an addivity is non-covalently associated with a hydrogel or hydrogel component.

In some embodiments, provided scaffolds include additives at a total amount from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, provided scaffolds include one or more additives at a molar ratio relative to a scaffold of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, provided scaffolds include therapeutic, preventative, and/or diagnostic additives, agents, and/or functional moieties. In some embodiments, a scaffold includes amino acids, antibodies or fragments or portions thereof, analgesics, anesthetics, angiogenesis inhibitors, antibodies, antibiotics or antimicrobial compounds, anticoagulants, antifungals, antigens, antihypertensives, antioxidants, antivirals, anti-cancer agents, anti-cholinergics, anti-depressants, anti-glaucoma agents, anti-inflammatory agents, anti-neoplastic agents, anti-psychotics, aptamers, β-adrenergic blocking agents, birth control agents, cardiovascular active agents, chemotherapeutic agents, cells, cytokines, decongestants, diuretics, drugs, dyes, enzymes, enzyme inhibitors, growth factors or recombinant growth factors and fragments and variants thereof, hormones, hormone antagonists, neuroprotectants, nucleic acids (DNA, RNA, siRNA), nucleic acid analogues, nucleotides, oligonucleotides, pharmacologic agents, peptides, peptide nucleic acids, prodrugs, progestational agents, proteins, sedatives, small molecules, steroidal agents, toxins, vaccines, vasoactive agents, vitamins, and combinations thereof.

Additives suitable for use with the present disclosure include biologically or pharmaceutically active compounds. Examples of biologically active compounds include, but are not limited to: cell attachment mediators, such as collagen, elastin, fibronectin, vitronectin, laminin, proteoglycans, or peptides containing known integrin binding domains e.g. ‘RGD’ integrin binding sequence, or variations thereof, that are known to affect cellular attachment (Schaffner P & Dard 2003 Cell Mol Life Sci. January; 60(1): 119-32; Hersel U. et al. 2003 Biomaterials. November; 24(24):4385-415); biologically active ligands; and substances that enhance or exclude particular varieties of cellular or tissue ingrowth. For example, the steps of cellular repopulation of the scaffold matrix preferably are conducted in the presence of growth factors effective to promote proliferation of the cultured cells employed to repopulate the matrix. Agents that promote proliferation will be dependent on the cell type employed. For example, when fibroblast cells are employed, a growth factor for use herein may be fibroblast growth factor (FGF), most preferably basic fibroblast growth factor (bFGF) (Human Recombinant bFGF, UPSTATE Biotechnology, Inc.). Other examples of additive agents that enhance proliferation or differentiation include, but are not limited to, osteoinductive substances, such as bone morphogenic proteins (BMP); cytokines, growth factors such as epidermal growth factor (EGF), platelet-derived growth factor (PDGF), insulin-like growth factor (IGF-I and II) TGF-β, and the like. As used herein, the term additive also encompasses antibodies, DNA, RNA, modified RNA/protein composites, glycogens or other sugars, and alcohols.

Cells

In some embodiments, provided scaffolds include cells. Cells suitable for use herein include, but are not limited to, progenitor cells or stem cells, smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

In some embodiments, provided scaffolds include organisms, such as, a bacterium, fungus, plant or animal, or a virus.

Nucleic Acids

In some embodiments, provided scaffolds include additives, for example, nucleic acid agents. In some embodiments, a hydrogel may release nucleic acid agents. In some embodiments, a nucleic acid agent is or includes a therapeutic agent. In some embodiments, a nucleic acid agent is or includes a diagnostic agent. In some embodiments, a nucleic acid agent is or includes a prophylactic agent.

It would be appreciate by those of ordinary skill in the art that a nucleic acid agent can have a length within a broad range. In some embodiments, a nucleic acid agent has a nucleotide sequence of at least about 40, for example at least about 60, at least about 80, at least about 100, at least about 200, at least about 500, at least about 1000, or at least about 3000 nucleotides in length. In some embodiments, a nucleic acid agent has a length from about 6 to about 40 nucleotides. For example, a nucleic acid agent may be from about 12 to about 35 nucleotides in length, from about 12 to about 20 nucleotides in length or from about 18 to about 32 nucleotides in length.

In some embodiments, nucleic acid agents may be or include deoxyribonucleic acids (DNA), ribonucleic acids (RNA), peptide nucleic acids (PNA), morpholino nucleic acids, locked nucleic acids (LNA), glycol nucleic acids (GNA), threose nucleic acids (TNA), and/or combinations thereof.

In some embodiments, a nucleic acid has a nucleotide sequence that is or includes at least one protein-coding element. In some embodiments, a nucleic acid has a nucleotide sequence that is or includes at least one element that is a complement to a protein-coding sequence. In some embodiments, a nucleic acid has a nucleotide sequence that includes one or more gene expression regulatory elements (e.g., promoter elements, enhancer elements, splice donor sites, splice acceptor sites, transcription termination sequences, translation initiation sequences, translation termination sequences, etc.). In some embodiments, a nucleic acid has a nucleotide sequence that includes an origin of replication. In some embodiments, a nucleic acid has a nucleotide sequence that includes one or more integration sequences. In some embodiments, a nucleic acid has a nucleotide sequence that includes one or more elements that participate in intra- or inter-molecular recombination (e.g., homologous recombination). In some embodiments, a nucleic acid has enzymatic activity. In some embodiments, a nucleic acid hybridizes with a target in a cell, tissue, or organism. In some embodiments, a nucleic acid acts on (e.g., binds with, cleaves, etc.) a target inside a cell. In some embodiments, a nucleic acid is expressed in a cell after release from a provided composition. In some embodiments, a nucleic acid integrates into a genome in a cell after release from a provided composition.

In some embodiments, nucleic acid agents have single-stranded nucleotide sequences. In some embodiments, nucleic acid agents have nucleotide sequences that fold into higher order structures (e.g., double and/or triple-stranded structures). In some embodiments, a nucleic acid agent is or includes an oligonucleotide. In some embodiments, a nucleic acid agent is or includes an antisense oligonucleotide. Nucleic acid agents may include a chemical modification at the individual nucleotide level or at the oligonucleotide backbone level, or it may have no modifications.

In some embodiments of the present disclosure, a nucleic acid agent is an siRNA agent. Short interfering RNA (siRNA) includes an RNA duplex that is approximately 19 basepairs long and optionally further includes one or two single-stranded overhangs. An siRNA may be formed from two RNA molecules that hybridize together, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

Short hairpin RNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

In describing siRNAs it will frequently be convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to include sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below may refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to shRNAs.

An siRNA agent is considered to be targeted to a target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. Since the effect of targeting a transcript is to reduce or inhibit expression of the gene that directs synthesis of the transcript, an siRNA, shRNA, targeted to a transcript is also considered to target the gene that directs synthesis of the transcript even though the gene itself (i.e., genomic DNA) is not thought to interact with the siRNA, shRNA, or components of the cellular silencing machinery. Thus in some embodiments, an siRNA, shRNA, that targets a transcript is understood to target the gene that provides a template for synthesis of the transcript.

In some embodiments, an siRNA agent can inhibit expression of a polypeptide (e.g., a protein). Exemplary polypeptides include, but are not limited to, matrix metallopeptidase 9 (MMP-9), neutral endopeptidase (NEP) and protein tyrosine phosphatase 1B (PTP1B).

Antibodies

In some embodiments, provided scaffolds include additives, for example, antibodies. Suitable antibodies for incorporation in hydrogels include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

Proteins

In some embodiments, provided scaffolds include additives, for example, polypeptides (e.g., proteins), including but are not limited to: one or more antigens, cytokines, hormones, chemokines, enzymes, and any combination thereof as an agent and/or functional group. Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

Growth Factor

In some embodiments, provided scaffolds include additives, for example, growth factor. In some embodiments, a hydrogel may release growth factor. In some embodiments, a hydrogel may release multiple growth factors. In some embodiments growth factor known in the art include, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof.

In some embodiments, provided scaffolds include additives, for example, that are particularly useful for healing. Exemplary agents useful as growth factor for defect repair and/or healing can include, but are not limited to, growth factors for defect treatment modalities now known in the art or later-developed; exemplary factors, agents or modalities including natural or synthetic growth factors, cytokines, or modulators thereof to promote bone and/or tissue defect healing. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that includes both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine A1 agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination. More details can be found in U.S. Pat. No. 8,247,384, the contents of which are incorporated herein by reference.

It is to be understood that agents useful for growth factor for healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the wound healing agent, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

Therapeutics

Antibiotics

In some embodiments, provided scaffolds include additives, for example, antibiotics. Antibiotics suitable for incorporation in hydrogels include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, β-lactam antibiotics, rifamycins, novobiocin, fusidate sodium, capreomycin, colistimethate, gramicidin, doxycycline, erythromycin, nalidixic acid, and vancomycin. For example, β-lactam antibiotics can be aziocillin, aztreonam, carbenicillin, cefoperazone, ceftriaxone, cephaloridine, cephalothin, moxalactam, piperacillin, ticarcillin and combination thereof.

Anti-Inflammatories

In some embodiments, provided scaffolds include additives, for example, anti-inflammatories. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Small Molecules

In some embodiments, an additive is or includes one or more therapeutic agents. In general, a therapeutic agent is or includes organic compounds or small molecules with pharmaceutical activity (e.g., activity that has been demonstrated with statistical significance in one or more relevant pre-clinical models or clinical settings). In some embodiments, a therapeutic agent is a clinically-used drug.

Diagnostics

In some embodiments, provided scaffolds include additives, for example, that are particularly useful as diagnostic agents. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MM include gadolinium chelates, as well as iron, magnesium, manganese, copper, and chromium. Examples of materials useful for CAT and x-ray imaging include iodine-based materials.

In some embodiments, provided scaffolds include additives, for example, radionuclides that are particularly useful as therapeutic and/or diagnostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming thermally-responsive conjugates in accordance with the invention include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pd, ¹⁰⁹Pd, ¹¹¹In, ⁶⁷Ga, ⁶⁸Ga, ⁷⁵Br, ⁷⁷Br, ⁹⁹mTc, ¹⁴C, ¹³N, ¹⁵O, ³²P, ³³P, and ¹⁸F. In some embodiments, a diagnostic agent may be a fluorescent, luminescent, or magnetic moiety.

Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Application Publication No.: 2004/0067503; Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site; both of which are incorporated herein by reference).

Others

In some embodiments, provided scaffolds include additives, for example, particularly useful for wound healing. In some embodiments, agents useful for wound healing include stimulators, enhancers or positive mediators of the wound healing cascade which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

In some embodiments, an active agent may include or be selected from neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

In some embodiments, provided scaffolds include additives, for example, an optically or electrically active agent, including but not limited to, chromophores; light emitting organic compounds such as luciferin, carotenes; light emitting inorganic compounds, such as chemical dyes; light harvesting compounds such as chlorophyll, bacteriorhodopsin, protorhodopsin, and porphyrins; light capturing complexes such as phycobiliproteins; and related electronically active compounds; and combinations thereof.

### Methods of Forming Hydrogels

Silk fibroin sol-gel transition occurs through inter-molecular and intra-molecular interactions (mainly formation of hydrogen bonds and hydrophobic interactions) among protein chains, which fold from amorphous to thermodynamically stable β-sheets, driven by exposure of silk solutions to shear forces, electric fields, pH near or below the isoelectric point (pI=3.8-3.9), polar solvents, heat and water removal. (See U. J. Kim, 5 Biomacromolecules, 786-92 (2004) and S. Nagarkar, 12 Phys. Chem. Chem. Phys., 3834-44 (2010) herein incorporated by reference in their entirety). The soft-micelle assembly process is also regulated by the strong amphiphilic (hydrophobic and hydrophilic domains) nature of the protein, where short hydrophilic (amorphous) spacers intervene between large hydrophobic (crystallizable) blocks and play a critical role in preventing premature β-sheet formation and in modulating water solubility. (See H. J. Jin, 424 Nature, 1057-61 (2003) herein incorporated by reference in its entirety).

Development of inter-molecular bonds results in aggregation of silk fibroin micelles into interconnected micron-sized particles with progressive loss of transparency of the silk solution, ultimately becoming a white hydrogel due to light scattering. Despite numerous applications of scaffolds in biomedical engineering, the lack of transparency has been of hindrance to fully capitalize on this material format. (See M. Choi, 7 Nat. Photonics, 987-994 (2013) herein incorporated by reference in its entirety). For example, biological entities (e.g. cells), light sensitive molecules (e.g. fluorescent, bioluminescent, photoactive macromolecules) and optogenetic tools can be incorporated into hydrogels for sensing and diagnostic applications, to generate biomimetic biological systems or to build optical interfaces with living tissues. Transparency is also the main characteristic of cornea tissue where silk fibroin has shown potential as scaffolding material for cornea replacements. (See T. Chirila, 561-565 Materials Science Forum, 1549-1552 (2007), T. V. Chirila, 14 Tissue Eng Part A, 1203-11 (2008), K. Higa, 27 Cornea, Suppl 1, S41-7 (2008), E. S. Gil, 10 Macromol. Biosci., 664-73 (2010), J. Wu, 35 Biomaterials, 3744-55 (2014), B. D. Lawrence, 8 Acta Biomater., 3732-3743 (2012), E. S. Gil, 31 Biomaterials, 8953-63 (2010), B. D. Lawrence, 30 Biomaterials, 1299-308 (2009) herein incorporated by reference in their entirety).

In some embodiments, fabrication of scaffolds as provided herein includes providing silk fibroin. In some embodiments, providing silk fibroin includes providing silk cocoons; boiling the silk cocoons in 0.02 M Na₂CO₃ to remove outer layers of sericin; cooling and unraveling cocoons into fibroin fibers; solubilizing fibers in a highly concentrated solution of chaotropic ions (LiBr); dialysizing the solution to remove the chaotropic salts from the solution, yielding a pure fibroin solution. Silk fibroin in solution possesses an amorphous structure (mostly random coils) and is arranged in micelles. In some embodiments, methods of forming scaffolds further includes mixing silk fibroin solution with acetone to form freestanding scaffolds. When the silk fibroin solution was exposed to polar solvents, a combination of amorphous-to-crystalline conformational changes together with aggregation results in the formation of silk particles, which arrange together in the presence of water forming a freestanding hydrogel structure.

### Multiphoton Micromachining Methods

In some embodiments, scaffolds as disclosed herein are manufactured as described herein. In some embodiments, methods of providing, preparing, and/or manufacturing scaffolds of the present disclosure utilize a multiphoton absorption process.

In some embodiments, when provided scaffolds are exposed to focused light, they absorb localized heat at a focal spot. In some embodiments, when absorbing localized heat at a focal spot scaffold material, such a hydrogel is removed. In some embodiments, when hydrogel is removed, a cavity is formed.

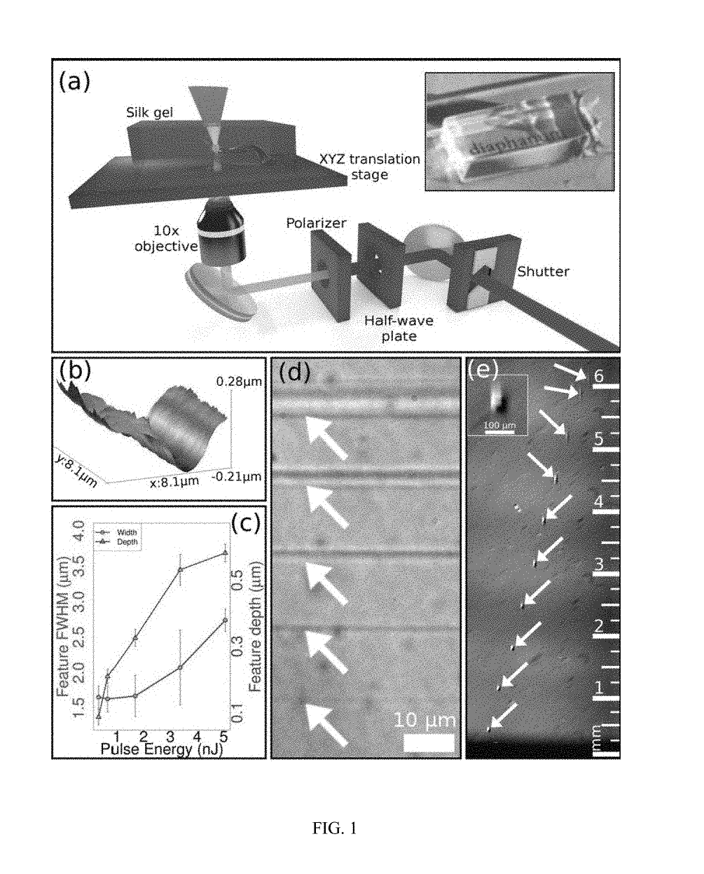

Previous work involving photomodification of silk has thus far only considered surface modification of dried films. (See Lazare S, et al. Bombyx mori silk protein films microprocessing with a nano-second ultraviolet laser and a femtosecond laser workstation: Theory and experiments”, 106 Appl Phys, A Mater Sci Process, 67-77 (2011)). In some embodiments, silk fibroin hydrogels are ideally suited to multiphoton laser micromachining (FIG. 1 at panel (A) at the inset). (See Partlow B P, et al. “Highly tunable elastomeric silk biomaterials”, 24 Adv Funct Mater 29, 4615-4624 2014). In some embodiments, silk fibroin hydrogels are robust enough to be easily handled, amenable to cell growth, and well tolerated upon implantation. In some embodiments, silk fibroin hydrogels are greater than 90% water, which allows material disrupted during MPA to be deposited around the outside of the machined region without fouling.

Multiphoton Absorption occurs under extremely intense illumination where two or more low-energy photons are absorbed simultaneously by a material. (See Goppert-Mayer M., “Elementary processes with two quantum jumps”, 9 Ann Phys, 273-294 (1931)). To achieve photon densities high enough for MPA, very short laser pulses must be tightly focused within a material.

If the material is transparent to the low-energy photons, very little of the light is absorbed at the surface, allowing a focal spot to be formed, and WA to occur, deep within the material.

In some embodiments, multiphoton-induced structural modification leads to formation of cavities below its surface.

In prior systems, transparent materials exhibited very high threshold power requirements that necessitated the use of high numerical aperture objectives, or amplified femtosecond pulses to initiate MPA for formation of cavities below its surface. Extremely high light intensities found in these amplified pulses can locally change a material's refractive index, resulting in self-focusing of the beam. Generally, self-focusing limits the depth at which a tight focal spot can be formed. MPA-induced formation of cavities below its surface has been limited to less than 200 μm below the surface of the material. (See Oujja M, et al., “Three dimensional microstructuring of biopolymers by femtosecond laser irradiation”, 95 Appl Phys Lett 263703 (2009)).

The present disclosure encompasses a recognition certain materials a more efficient multiphoton absorbers. The present disclosure also encompasses a recognition certain materials having a large multiphoton cross-section would allow the initiation of MPA at low threshold powers. Without wishing to be bound to a particular theory, it is believed that large multiphoton cross-section would potentially reduce the effects of self-focusing.

The present disclosure includes methods of 3D, multiscale laser machining of soft, transparent biomaterials that are suited for cellular growth and/or implantation.

In some embodiments, provided methods utilize ultrafast laser to generate high-resolution, 3D structures within a bulk of a transparent soft-biomaterial formulation that can support cell growth and allow cells to penetrate deep within such a material. It is believed that structures may be created by multiphoton absorption deep below a surface of a material. In some embodiments, structures may be formed more than a cm beneath a surface, which represents a depth of about ten greater than prior methods. An ability to create micrometer-scale cavities below its surface over such a large volume has promising biomedical applications.

In some embodiments, the present disclosure provides for inducing formation cavities beneath a surface of a scaffold.

In some embodiments, for example, with silk fibroin hydrogels, a step of irradiating is characterized by low-energy. In some embodiments, pulse energy is less than about 0.25 nJ per pulse, less than about 0.5 nJ per pulse, less than about 0.6 nJ per pulse, less than about 0.7 nJ per pulse, less than about 0.8 nJ per pulse, less than about 0.9 nJ per pulse, less than about 1.0 nJ per pulse, less than about 1.1 nJ per pulse, less than about 1.2 nJ per pulse, less than about 1.3 nJ per pulse, less than about 1.4 nJ per pulse, less than about 1.5 nJ per pulse, less than about 1.6 nJ per pulse, less than about 1.7 nJ per pulse, less than about 1.8 nJ per pulse, less than about 1.9 nJ per pulse, less than about 2.0 nJ per pulse, less than about 2.1 nJ per pulse, less than about 2.2 nJ per pulse, less than about 2.3 nJ per pulse, less than about 2.4 nJ per pulse, less than about 2.5 nJ per pulse, less than about 2.6 nJ per pulse, less than about 2.7 nJ per pulse, less than about 2.8 nJ per pulse, less than about 2.9 nJ, less than about 3.0 nJ, less than about 3.1 nJ per pulse, less than about 3.2 nJ per pulse, less than about 3.3 nJ per pulse, less than about 3.4 nJ per pulse, less than about 3.5 nJ per pulse, less than about 3.6 nJ per pulse, less than about 3.7 nJ per pulse, less than about 3.8 nJ per pulse, less than about 4.0 nJ, less than about 4.1 nJ per pulse, less than about 4.2 nJ per pulse, less than about 4.3 nJ per pulse, less than about 4.4 nJ per pulse, less than about 4.5 nJ per pulse, less than about 4.6 nJ per pulse, less than about 4.7 nJ per pulse, less than about 4.8 nJ per pulse, less than about 4.9 nJ per pulse, less than about 5.0 nJ per pulse, less than about 6.0 nJ per pulse, less than about 7.0 nJ per pulse, less than about 8.0 nJ per pulse, less than about 9.0 nJ per pulse, less than about 10.0 nJ per pulse, less than about 11.0 nJ per pulse, less than about 12.0 nJ per pulse, less than about 13.0 nJ per pulse, less than about 14.0 nJ per pulse, less than about 15.0 nJ per pulse, less than about 20.0 nJ per pulse, less than about 25.0 nJ per pulse, less than about 30.0 nJ per pulse, less than about 35.0 nJ per pulse, less than about 40.0 nJ per pulse, less than about 45.0 nJ per pulse, less than about 50.0 nJ per pulse, less than about 75.0 nJ per pulse, less than about 100 nJ per pulse, less than about 200 nJ per pulse, less than about 300 nJ per pulse, less than about 400 nJ per pulse, less than about 500 nJ per pulse, less than about 600 nJ per pulse, less than about 700 nJ per pulse, less than about 800 nJ per pulse, less than about 800 nJ per pulse, less than about 900 nJ per pulse, or less than about a micro-joule per pulse.

In some embodiments, a step of irradiating is characterized by low power. In some embodiments, pulse power is less than 1 mW per pulse, less than 1 mW per pulse, less than 2 mW per pulse, less than 3 mW per pulse, less than 4 mW per pulse, less than 5 mW per pulse, less than 6 mW per pulse, less than 7 mW per pulse, less than 8 mW per pulse, less than 9 mW per pulse, less than 10 mW per pulse, less than 11 mW per pulse, less than 12 mW per pulse, less than 13 mW per pulse, less than 14 mW per pulse, less than 15 mW per pulse, less than 16 mW per pulse, less than 17 mW per pulse, less than 18 mW per pulse, less than 19 mW per pulse, less than 20 mW per pulse, less than 30 mW per pulse, less than 40 mW per pulse, less than 50 mW per pulse, less than 60 mW per pulse, less than 70 mW per pulse, less than 80 mW per pulse, less than 90 mW per pulse, less than 100 mW per pulse, less than 200 mW per pulse, less than 300 mW per pulse, less than 400 mW per pulse, less than 500 mW per pulse, less than 600 mW per pulse, less than 700 mW per pulse, less than 800 mW per pulse, less than 900 mW per pulse, or less than 1 W per pulse.

In some embodiments, a step of irradiating is characterized by a wavelength. In some embodiments, wavelength is about 200 nm to about 1750 nm. In some embodiments, an irradiating wavelength is any wavelength where a scaffold material is transparent. In some embodiments, a wavelength is about within the visual spectrum. In some embodiments, a wavelength is between about 380 nm and about 750 nm.

In some embodiments, wavelength is about 200 nm to about 400 nm. In some embodiments, wavelength is about 400 nm to about 600 nm. In some embodiments, wavelength is about 600 nm to about 800 nm. In some embodiments, wavelength is about 800 nm to about 1000 nm. In some embodiments, wavelength is about 1000 nm to about 1200 nm. In some embodiments, wavelength is about 1200 nm to about 1400 nm. In some embodiments, wavelength is about 1400 nm to about 1600 nm. In some embodiments, wavelength is about 1600 nm to about 1800 nm.

In some embodiments, wavelength is about 200 nm, about 250 nm, 300 nm, about 350 nm, about 360 nm, about 370 nm, about 380 nm, about 390 nm, 400 nm, about 410 nm, about 420 nm, about 430 nm, about 440 nm, about 450 nm, about 460 nm, about 470 nm, about 480 nm, about 490 nm, 500 nm, about 510 nm, about 520 nm, about 530 nm, about 540 nm, about 550 nm, about 560 nm, about 570 nm, about 580 nm, about 590 nm, 600 nm, about 610 nm, about 620 nm, about 630 nm, about 640 nm, about 650 nm, about 660 nm, about 670 nm, about 680 nm, about 690 nm, 700 nm, about 710 nm, about 720 nm, about 730 nm, about 740 nm, about 750 nm, 800 nm, about 850 nm, 900 nm, about 950 nm, 1000 nm, about 1050 nm, 1100 nm, about 1150 nm, 1200 nm, about 1250 nm, 1300 nm, about 1350 nm, 1400 nm, about 1450 nm, 1500 nm, about 1550 nm, 1600 nm, about 1650 nm, 1700 nm, or about 1750 nm.

In some embodiments, a step of irradiating is characterized in that it is as a pulsed beam. In some embodiments, a pulse beam is characterized in that it has a short pulse duration. In some embodiments, a pulse duration is on an order of femtoseconds. In some embodiments, a pulse duration is on an order of picoseconds. In some embodiments, a pulse duration is on an order of nanoseconds. In some embodiments, a pulse duration is on the order of microseconds. In some embodiments, a pulse duration is on the order of attoseconds.

In some embodiments, a step of irradiating is characterized in that it is as a pulsed beam. In some embodiments, a pulsed beam has a frequency of about 1 Hz to about 100 GHz. In some embodiments, a pulsed beam has a frequency of about 50 MHz. to about 100 MHz.

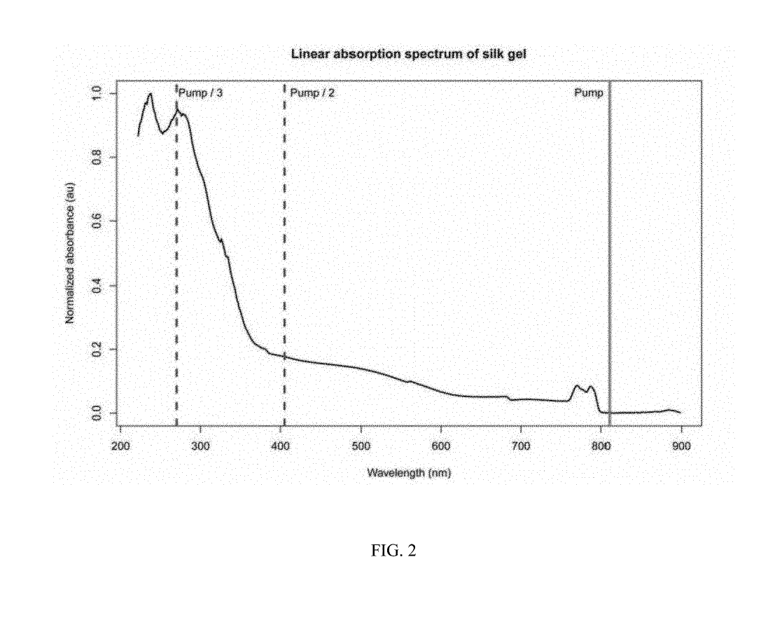

In some embodiments, for example, with silk fibroin hydrogels, relatively low-energy (sub-2 nJ per pulse) infrared (λ=810 nm) pulses at a high repetition rate (80 MHz) can be used to form cavities within these hydrogels in three dimensions. In some embodiments, for example, silk fibroin hydrogels have a linear absorption peak at 270 nm, suggesting this to be a three-photon absorption process. (See for example at FIG. 2).

In some embodiments, methods of providing, preparing, and/or manufacturing scaffolds of the present disclosure utilize a multiphoton absorption process.

In some embodiments, a multiphoton absorption process is characterized by a short time between pulses (12.5 ns). Without wishing to be bound to a theory, it is believed that the heat deposited by the first pulse that arrives does not have time to diffuse away before another pulse hits, leading to thermal accumulation at a focus of a beam which disrupts a silk structure thereby forming cavities, channels, openings, tunnels, vasculature, or voids. Such cavities formed survive handling, cell growth, and subdermal implantation.

In some embodiments, a laser pulse with a wavelength between about 200 nm and 1750 nm. In some embodiments, methods including irradiating a scaffold where the laser wavelength overlaps with a transmission profile of a scaffold, so that the scaffold is optically transparent at the irradiating wavelength.

In some embodiments, a laser pulse with a wavelength between about 200 nm and 1750 nm. In some embodiments, methods including irradiating a scaffold where the laser wavelength overlaps with a transmission profile of a scaffold, so that cells, additives, agents, and/or functional moieties are optically transparent at the irradiating wavelength.

In some embodiments, methods of providing, preparing, and/or manufacturing scaffolds of the present disclosure utilize a multiphoton absorption process include focusing a pulsed laser beam on a spot at a depth beneath a surface of a scaffold. In some embodiments, a spot is formed at a focal spot of laser light. In some embodiments, initiating multiphoton absorption forms at the hydrogel results in disrupting a scaffold at a spot. In some embodiments, disrupted scaffold is removed from a spot to form a cavity. While not wishing to be bound to any particular theory, it is believed that disrupted scaffold is removed by being vaporized, liquefied, ablated, moved, and or displaced by such a focused pulsed beam.

In some embodiments, irradiating forms cavities at a spot below a surface of a scaffold. In some embodiments, cavities are at least 20 μm, at least 30 μm, at least 40 μm, at least 50 μm, at least 60 μm, at least 70 μm, at least 80 μm, at least 90 μm, at least 100 μm, at least 110 μm, at least 120 μm, at least 130 μm, at least 140 μm, at least 150 μm, at least 160 μm, at least 170 μm, at least 180 μm, at least 190 μm, at least 200 μm, at least 210 μm, at least 220 μm, at least 230 μm, at least 240 μm, at least 250 μm, at least 260 μm, at least 270 μm, at least 280 μm, at least 290 μm, at least 300 μm, at least 325 μm, at least 350 μm, at least 375 μm, at least 400 μm, at least 425 μm, at least 450 μm, at least 475 μm, at least 500 μm, at least 525 μm, at least 550 μm, at least 575 μm, at least 600 μm, at least 625 μm, at least 650 μm, at least 675 μm, at least 700 μm, at least 725 μm, at least 750 μm, at least 775 μm, at least 800 μm, at least 825 μm, at least 850 μm, at least 875 μm, at least 900 μm, at least 925 μm, at least 950 μm, at least 975 μm, at least 1000 μm, at least 1025 μm, at least 1050 μm, at least 1075 μm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2.0 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3.0 mm, at least 3.1 mm, at least 3.2 mm, at least 3.3 mm, at least 3.4 mm, at least 3.5 mm, at least 3.6 mm, at least 3.7 mm, at least 3.8 mm, at least 3.9 mm, at least 4.0 mm, at least 4.1 mm, at least 4.2 mm, at least 4.3 mm, at least 4.4 mm, at least 4.5 mm, at least 4.6 mm, at least 4.7 mm, at least 4.8 mm, at least 4.9 mm, at least 5.0 mm, at least 6.0 mm, at least 7.0 mm, at least 8.0 mm, at least 9.0 mm, at least 10.0 mm, or more below a surface of a biomechanical scaffold.

In some embodiments, cavities in a provided biomechanical scaffold is characterized in that a width and length is approximately nano-scale to macro-scale. In some embodiments, cavities have a diameter of about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 1.3 μm, 1.4 μm, 1.5 μm, 1.6 μm, 1.7 μm, 1.8 μm, 1.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.5 μm, 11.0 μm, 11.5 μm, 12.0 μm, 12.5 μm, 13.0 μm, 13.5 μm, 14.0 μm, 14.5 μm, 15.0 μm, 15.5 μm, 16.0 μm, 16.5 μm, 17.0 μm, 17.5 μm, 18.0 μm, 18.5 μm, 19.0 μm, 19.5 μm, 20 μm, 21 μm, 22 μm, 23 μm, 24 μm, 25 μm, 26 μm, 27 μm, 28 μm, 29 μm, 30 μm, 35 μm, 40 μm, 45 μm, 50 μm, 100 μm, or more.

In some embodiments, cavities in a provided biomechanical scaffold is characterized in that a width and length is approximately nano-scale to macro-scale. In some embodiments, cavities have an x-dimension or a y-dimension of about 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.1 μm, 2.2 μm, 1.3 μm, 1.4 μm, 1.5 μm, 1.6 μm, 1.7 μm, 1.8 μm, 1.9 μm, 3.0 μm, 3.1 μm, 3.2 μm, 3.3 μm, 3.4 μm, 3.5 μm, 3.6 μm, 3.7 μm, 3.8 μm, 3.9 μm, 4.0 μm, 4.1 μm, 4.2 μm, 4.3 μm, 4.4 μm, 4.5 μm, 4.6 μm, 4.7 μm, 4.8 μm, 4.9 μm, 5.0 μm, 5.1 μm, 5.2 μm, 5.3 μm, 5.4 μm, 5.5 μm, 5.6 μm, 5.7 μm, 5.8 μm, 5.9 μm, 6.0 μm, 7.1 μm, 7.2 μm, 7.3 μm, 7.4 μm, 7.5 μm, 7.6 μm, 7.7 μm, 7.8 μm, 7.9 μm, 8.0 μm, 8.1 μm, 8.2 μm, 8.3 μm, 8.4 μm, 8.5 μm, 8.6 μm, 8.7 μm, 8.8 μm, 8.9 μm, 9.0 μm, 9.1 μm, 9.2 μm, 9.3 μm, 9.4 μm, 9.5 μm, 9.6 μm, 9.7 μm, 9.8 μm, 9.9 μm, 10.0 μm, 10.5 μm, 11.0 μm, 11.5 μm, 12.0 μm, 12.5 μm, 13.0 μm, 13.5 μm, 14.0 μm, 14.5 μm, 15.0 μm, 15.5 μm, 16.0 μm, 16.5 μm, 17.0 μm, 17.5 μm, 18.0 μm, 18.5 μm, 19.0 μm, 19.5 μm, 20 μm, 21 μm, 22 μm, 23 μm, 24 μm, 25 μm, 26 μm, 27 μm, 28 μm, 29 μm, 30 μm, 35 μm, 40 μm, 45 μm, 50 μm, 100 μm, or more.

In some embodiments, methods include irradiating at a power of less than would result in self-focusing. In some embodiments, polymers or proteins have a large multiphoton cross-section, such that they have a large maximum machining depth, which allows low-powered pulses to be used to initiate MPA without significant self-focusing.

In some embodiments, light penetrates a polymer and/or protein and is capable of penetrating such a polymer and/or protein to a depth below a surface of at least about 150 μm, at least about 200 μm, at least about 200 μm, at least about 210 μm, at least about 220 μm, at least about 230 μm, at least about 240 μm, at least about 250 μm, at least about 260 μm, at least about 270 μm, at least about 280 μm, at least about 290 μm, at least about 300 μm, at least about 310 μm, at least about 320 μm, at least about 330 μm, at least about 340 μm, at least about 350 μm, at least about 360 μm, at least about 370 μm, at least about 380 μm, at least about 390 μm, at least about 400 μm, at least about 410 μm, at least about 420 μm, at least about 430 μm, at least about 440 μm, at least about 450 μm, at least about 460 μm, at least about 470 μm, at least about 480 μm, at least about 490 μm, at least about 500 μm, at least about 600 μm, at least about 700 μm, at least about 800 μm, at least about 900 μm, at least about 1000 μm, at least about 1500 μm, at least about 2500 μm, at least about 5000 μm, or more.

In some embodiments, MPA may be used to form cavities beneath a surface of a protein and/or polymer. In some embodiments, voids have shape. In some embodiments, cavities are characterized by their shape. In some embodiments, cavities are characterized by their shape within a scaffold. In some embodiments, cavities have dimensions in an x and y axes.

In some embodiments, shapes beneath a surface of a protein and/or polymer are formed by a moving focus through such a material. In some embodiments, polymer and/or protein material is removed by MPA void formation and a focus passes through such a material.

In some embodiments, shapes beneath a surface of a protein and/or polymer are formed by a moving focus through such a material.

### EXEMPLIFICATION

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

The present Example describes an exemplary MPA workstation.

### Materials and Methods

Approximately 100 fs pulses of 810 nm light from a titanium sapphire oscillator (Tsunami, Spectra Physics) at a repetition rate of 80 MHz were passed through a computer-controlled shutter and directed into the rear accessory port of an inverted microscope. The light was focused to an about 5 μm spot through a 0.3 NA microscope objective with a working distance of 1.03 cm onto the sample, which was placed on a computer-controlled XYZ translation stage (Ludl Electronics). Using a custom LabView application, complex patterns could be micromachined by inputting stacks of binary images into the program. Pulse energies were manually adjusted via a half-wave plate and polarizer giving continuous control of pulse energy from 0.1 to about 10 nJ per pulse. FIG. 1 at panel (A) shows a custom-built three-dimensional laser writing workstation for multiphoton micromachining of biopolymer gels.

### Example 2

The present Example describes Hydrogel Preparation.

Silk fibroin was extracted as previously described and with a degumming time of 60 min. (See for example, Rockwood D N, et al., “Materials Fabrication from Bombyx mori Silk Fibroin”, 6 Nat Protoc 10, 1612-1631 (2011)). Gels were prepared by adding 10 units/mL type VI horseradish peroxidase and 10 μL/mL 1% hydrogen peroxide. (See for example, Partlow B P, et al., “Highly Tunable Elastomeric Silk Biomaterials”, 24 Adv Funct Mater 29, 4615-4624 (2014)). To facilitate fluorescence imaging, Rhodamine B-stained gels were prepared after the desired pattern had been micromachined. These gels were soaked in a solution of 0.1 mM Rhodamine B for 4 hours and then were rinsed in 10 changes of deionized water over the following 24 hours to remove any Rhodamine not bound to the silk.

### Example 3

The Present Example Describes Gel Micromachining.

Lines were micromachined on the top surface of a thin gel at pulse energies ranging from 0.25 to 5 nJ per pulse and were imaged on an MFP-three-dimensional-Bio AFM (Asylum Research). The samples were scanned in contact mode under PBS solution using TR800PSA cantilevers with a calibrated spring constant of 0.4 N/m.

Maximum depth of machining was determined by forming a silk gel inside a plastic fluorescence cuvette. Thirty μm thick lines were micromachined in the silk at regular depth intervals. Translation speed varied between 100 and 25 μm/s depending on the depth. A side view of the lines was obtained by rotating the cuvette 90° and imaging via bright-field microscopy.

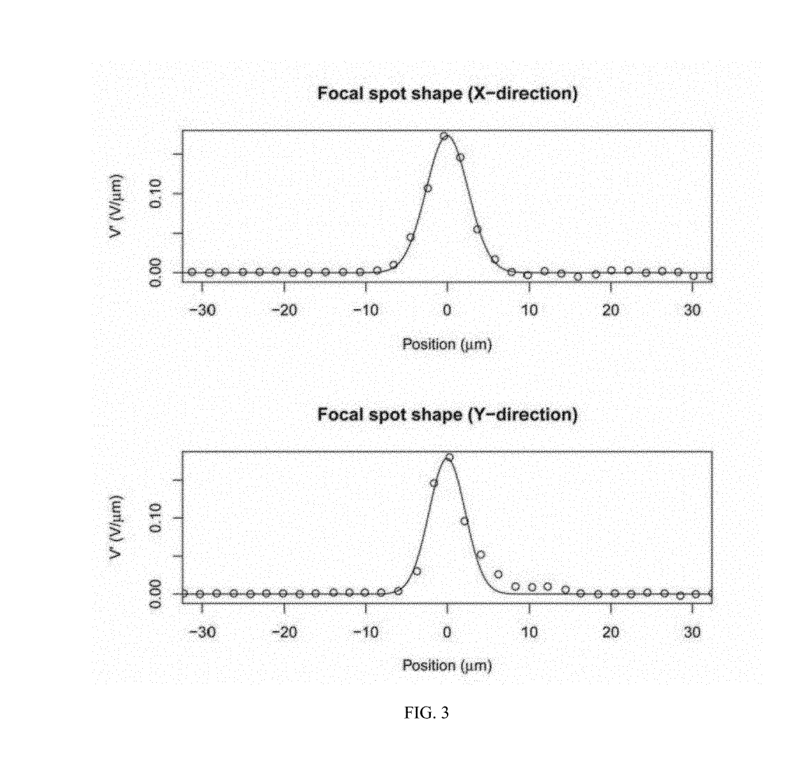

FIG. 3 shows beam profiles of ultrashort (about 100 fs) laser pulses at a pulse repetition frequency of 80 MHz that were focused into a bulk of a silk hydrogel using a 10× (NA=0.3) microscope objective. The sample was mounted on a three-axis micropositioning stage. The sample could then be moved so the beam was focused in different locations within the material. Generation of complex three-dimensional patterns within the material was achieved by computer control over the stage translation.

FIG. 1 at panel (D) shows the relationship between pulse energy and void size, which was characterized by micromachining a series of lines on the top surface of a gel about 1 mm thick. Each line was made by a single pass of the laser at a constant speed of 50 μm/s with varying pulse energies. After machining, the lines were imaged via atomic force microscopy (AFM). Structural changes were observed in the silk gel at pulse energies above about 0.25 nJ per pulse. At this power, the average trench dimensions were 1.5 μm full width at half maximum (FWHM) in width and 100 nm in depth. FIG. 1 at panel (C) shows that the dimensions increased to 2.5 μm FWHM and 600 nm in depth when the pulse energy was raised to 5 nJ. FIG. 1 at panel (B) shows AFM measurements that confirmed that the change in appearance of the machined region was due to material removal and not local changes in refractive index.

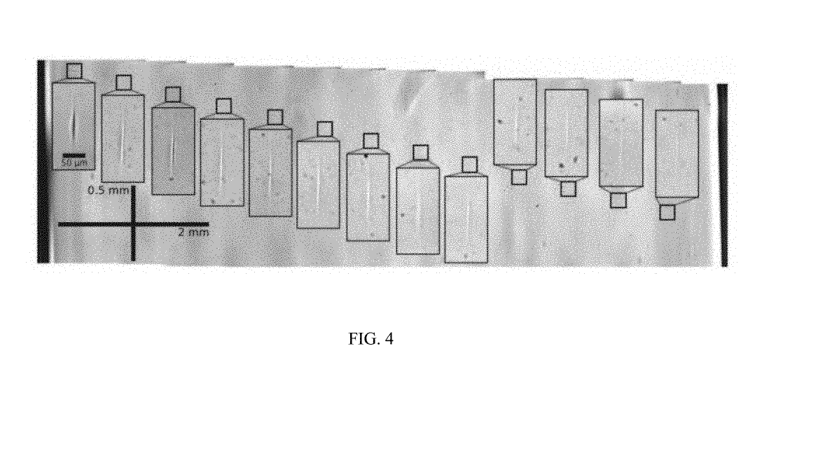

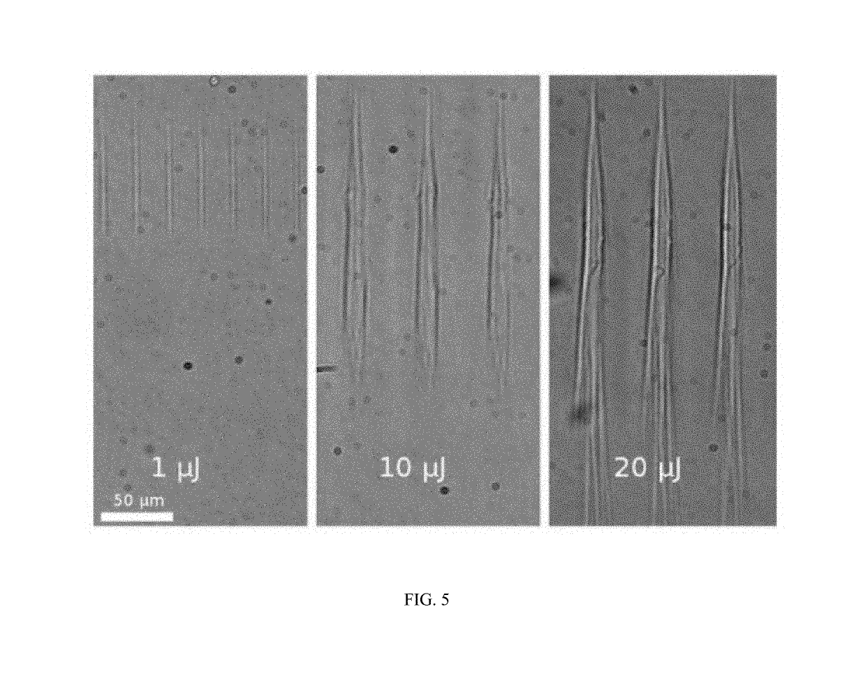

The depth at which features could be micromachined was tested by forming a gel inside a plastic fluorescence cuvette. Features were micromachined inside the gel at various depths and subsequently imaged by rotating the cuvette 90° and examining the features using bright-field microscopy. FIG. 1 at panel (E) and FIG. 4 show visible features that were found in the gel up to 8 mm below its surface. Deeper features should be possible using a longer working distance objective with a similar numerical aperture. FIG. 4 shows a large maximum machining depth. While not wishing to be bound to a particular theory, it is believed that silk's optical clarity and a large multiphoton cross-section of the protein, allows low-powered pulses to be used to initiate MPA without significant self-focusing. FIG. 5 shows amplified laser pulses that were used to create voids. It is estimated that the critical power for self-focusing for silk hydrogels is greater than 6 MW, which is more than 100× more power than is found in the pulses used for multiphoton micromachining as shown in FIG. 5. This combination of qualities is, to the best of our knowledge, unique to silk and enables multiphoton micromachining to occur at such large depths. Deep, high-resolution features such as these, combined with the ability to dope the silk with growth factors and other compounds, could be used for the generation of complex three-dimensional patterned cell scaffolds to form microenvironments for different cell types within the same scaffold.

With maximum penetration depth of nearly 1 cm and a lateral resolution on the order of 5 silk hydrogels are an excellent substrate for multiphoton micromachining. Given the limits of travel of the micropositioning stage, the total addressable volume of our workstation was greater than 100 cm³. Within this volume, individual voxels as small as 125 μm³ could be removed at will, with the removed material deposited along the outer edges of the machined regions.

### Example 4

The Present Example Describes Two-Dimensional Contact Guidance.

Silk solutions were filtered through a 0.22 μm filter and gelled in a 35 mm Petri dish. Before removal from the hood, dishes were sealed with parafilm to maintain sterility. Lines were then micromachined onto the top surface of the gel. Human foreskin fibroblasts were seeded onto the gel and cultured in DMEM with 10% FBS at 37° C., 5% CO₂. Gels were imaged via phase contrast microscopy at day 1, 3, and 5 post-seeding. On day 8 the cells were stained with a Live/DeadViability/Cytotoxicity kit (Molecular Probes, Inc.) fluorescence assay and imaged via fluorescence microscopy.

### Example 5

The Present Example Describes MPA in Cell-Laden Hydrogels.

Human mesenchymal stem cells (hMSCs) were isolated from fresh bone marrow aspirate (Lonza) as previously described. (See for example, Altman G H, et al., “Cell Differentiation by Mechanical Stress”, 16 FASEB J 2, 270-272 (2002)). hMSCs were gently mixed into a partially gelled silk hydrogel at a rate of 1,000 cells per mm³. One hundred μL of the silk/cell mixture was added to each glass-bottomed Petri dish. (See for example, Partlow B P, et al., “Highly Tunable Elastomeric Silk Biomaterials”, 24 Adv Funct Mater 29, 4615-4624 (2014)). After gelation, micromachining was performed on the cell-laden hydrogels. Within 4 hours of machining the cells were stained with a Live/DeadViability/Cytotoxicity kit (Molecular Probes, Inc.) and examined via confocal fluorescence microscopy.

### Example 6

The Present Example Describes Three-Dimensional Contact Guidance.

Sterile gels were prepared as described in Two-dimensional Contact Guidance above. Three-dimensional Y-shaped branching patterns were machined into the gel, with the main branch of the Y intersecting the top surface of the gel. Main branch diameters were 200 μm and 400 μm in the small and large features, respectively. Human foreskin fibroblasts were seeded onto the surface of the gel after micromachining. Gels were examined via confocal microscopy on day 5, 9, and 14 post-seeding. The day before each imaging session dishes were stained with CytoTraker Green (Molecular Probes).

### Example 7

The Present Example Describes Implantation.

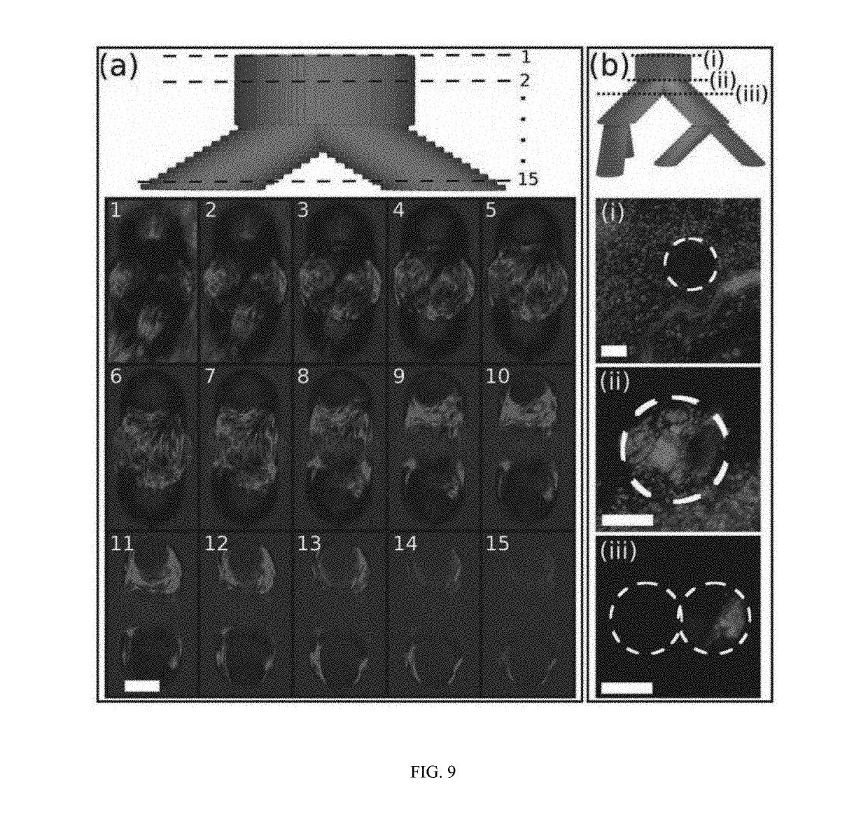

Sterile gels were prepared in 35 mm Petri dishes as described above and a 4 mm biopsy punch was used to remove cylinders of gel. FIG. 9 at panel (B) shows branching patterns machined into each cylinder. All procedures involving mice were approved by the Tufts University Institutional Animal Care and Use Committee. Animals were anesthetized by isoflurane inhalation during the procedure. Machined gels were implanted s.c. into the lumbar region of three mice. Silk implants and adjacent tissues were extracted following euthanasia (carbon dioxide asphyxiation) at 2 week, 3 week, and 4 week post-implantation. Gels were recovered from the mice and fixed in 10% formalin and stained with Phalloidin and DAPI.

### Example 8

The Present Example Describes Tissue Formation.

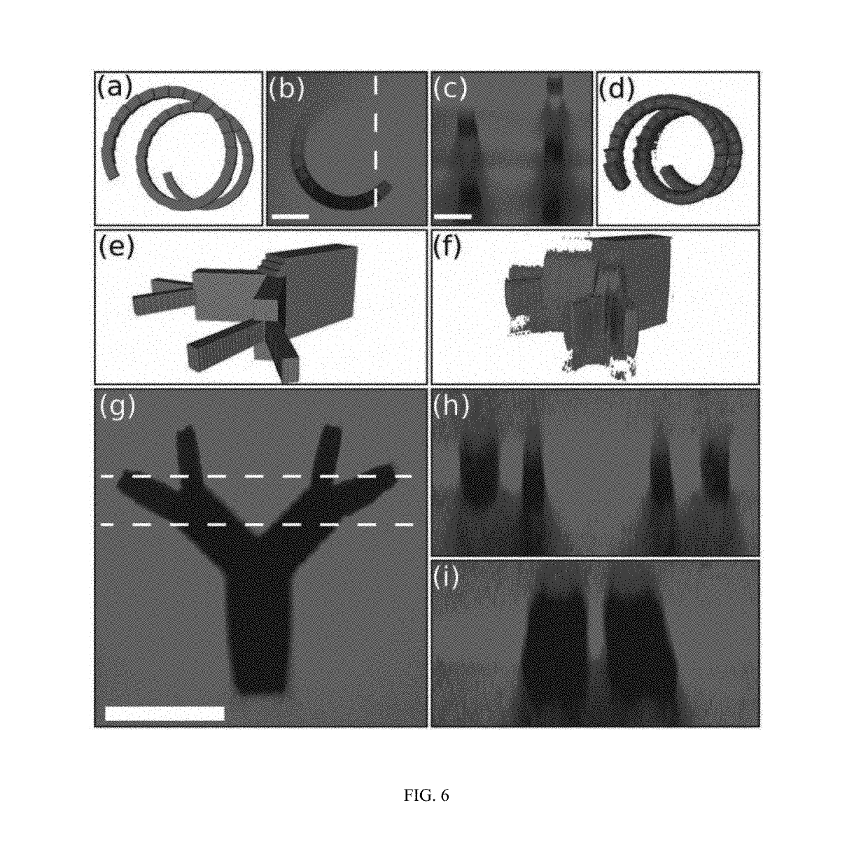

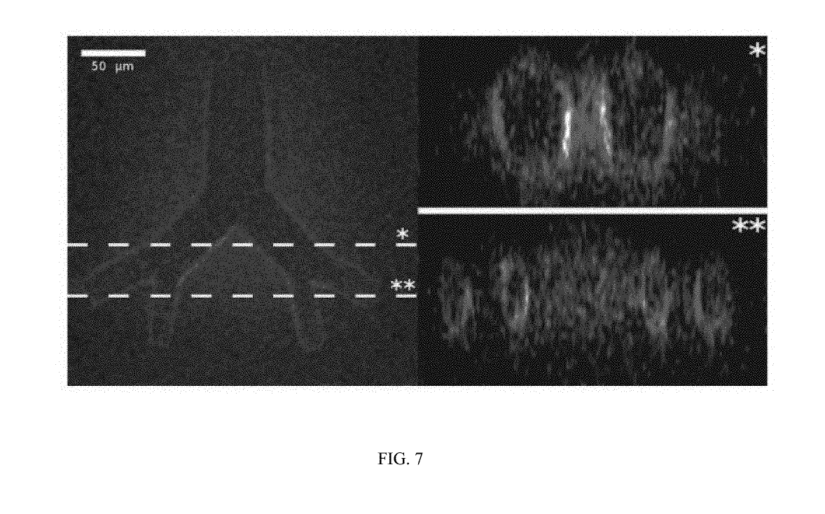

To explore the practicality of this technique to generate complex three-dimensional structures, test patterns were micromachined into the bulk of the silk gel. FIG. 6 at panel (A) shows a first helix consisting of two turns with an outer diameter of 200 μm. The structure started roughly 500 μm below the surface and extended 400 μm further into the gel. FIG. 6 at panel (E) shows a second pattern, which was a blood vessel-like branching pattern. This structure was situated 300 μm from the surface and had a vertical extent of 100 μm. To image these patterns, the silk was stained with Rhodamine B after multiphoton micromachining and tomographic images were collected using confocal microscopy. The Rhodamine-stained silk fluoresced brightly whereas the machined regions were dark, indicating removal of the hydrogel in these regions. In most cases, the edges of the machined features showed evidence of greater material removal than the bulk of the features. This pattern was due to the control program, which paused lateral motion of the micropositioning stage at the end of each line before closing the shutter so the edges of the features were always exposed to more pulses than the center. Increased fluorescence was also visible around the edges of the features, which we attribute to the deposition of removed material along the borders. FIG. 7 shows deposition of removed material along the borders when imaging using the autofluorescence of silk for contrast rather than exogenous stains.

To be useful in biomedical applications, a material must be nontoxic and support cell growth. To ensure that the machined regions were not harmful to cells in culture, we prepared sterile gels by filtering the silk through a 0.22 μm pore filter and mixed the solution in a 35 mm diameter plastic Petri dish under sterile conditions for gelation. Before removing the dishes from the hood the lids were covered with parafilm to maintain sterility. All machining of the gels was done within the sealed Petri dishes in ambient conditions.

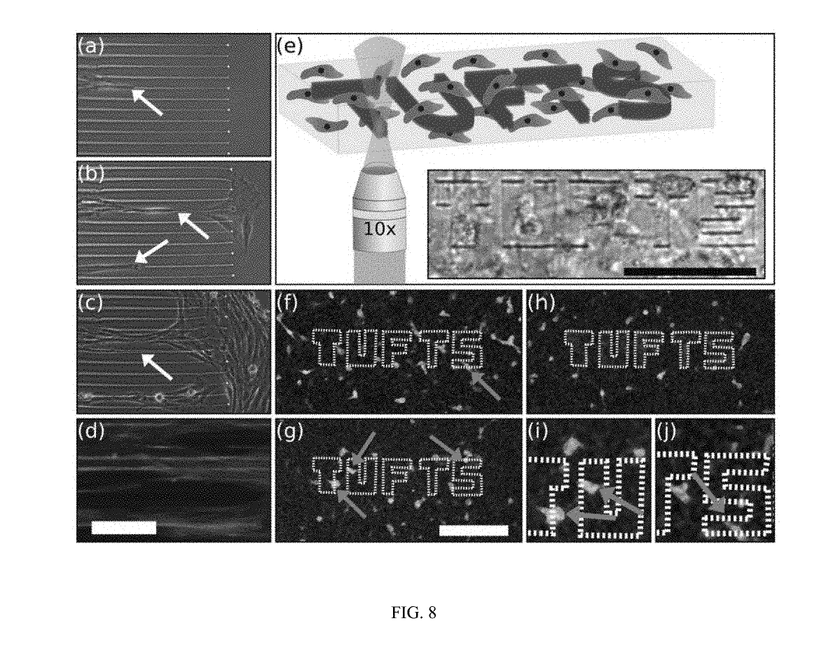

Parallel lines about 3 μm in width separated by about 20 μm were micromachined onto the top surface of a gel through the bulk. Human foreskin fibroblasts were seeded on the surface and observed using phase contrast microscopy as they attached and spread over the dish. FIG. 8 at panels (A)-(D) show that the cells tended to generally align with the grooves machined into the gel and grew parallel with these surface features. This contact guidance phenomenon is well-known and has previously been used to induce alignment of various cell types. (See for example, Dunn G A. and Ebendal T. “Contact Guidance on Oriented Collagen Gels”, 111 Exp Cell Res 2, 475-479 (1978); Gomez N., et al., “Polarization of Hippocampal Neurons with Competitive Surface Stimuli: Contact Guidance Cues are Preferred over Chemical Ligands”, 4 J R Soc Interface 13, 223-233 (2007)). Because features can be machined onto the gel through a sealed dish, we hypothesize that this could be a convenient method to reorient or disrupt already established cell cultures.

In tissue engineering, access to oxygen and nutrients within an artificial tissue is a major challenge that limits cell density within tissue engineered constructs. (See for example, Sachlos E. and Czernuszka J T. “Making Tissue Engineering Scaffolds Work. Review: The Application of Solid Freeform Fabrication Technology to the Production of Tissue Engineering Scaffolds”, 5 Eur Cell Mater, 29-39, discussion 39-40 (2003)). To address this issue, researchers have generated scaffolds with interconnected porous networks. (See for example, Bellan L M, et al., “Fabrication of an Artificial 3-Dimensional Vascular Network using Sacrificial Sugar Structures”, 5 Soft Matter, 1354-1357 (2009)). However, such pores are randomly distributed, limiting the amount of control of cell growth and infiltration that is possible. Multiphoton micromachining allows fully predetermined micrometer-scale features to be generated within a construct, allowing spatial control over cell infiltration. For example, vasculature for an organ may pre-mapped. Methods and technologies provided herein may then be used to form cavities according to such a map in a cell encapsulated scaffold, thereby forming an organ. FIG. 9 at panel (A) shows that micromachined features within the silk hydrogels could be used to direct cell growth in three dimensions, Y-shaped branching patterns were machined into the gels such that the main branch intersected the surface, allowing cells and media to penetrate the bulk of the gel. Cells were stained with a fluorescent dye and confocal images were taken of each feature at days 5, 9, and 14 post-seeding. Cell density was assessed at three locations within each feature: the main branch, the transition region, and the lower branch. By day 9 and continuing to day 14, cells were observed in all three regions in 100% of the small features. The larger features were less well populated with cells found in 100% of the main branches, 86% of the transition regions, and only 14% of the lower branches by day 9. On day 14, 71% of the large features had cells in the lower branches. One of the large features did not intersect the surface of the gel and was omitted from this analysis. FIG. 9 at panel (A) shows that no subsurface cells were observed in areas that were not laser machined.

Rather than providing a means for cells to infiltrate a material from the surface, it is often easier to encapsulate cells within the material itself. It has been shown that human mesenchymal stem cells (hMSCs) can be encapsulated within this type of silk hydrogel. (See for example, Partlow B P, et al., “Highly Tunable Elastomeric Silk Biomaterials”, 24 Adv Funct Mater 29, 4615-4624 (2014)). When cells are encapsulated in this way however, the concentrations of oxygen, nutrients, and growth factors are governed by diffusion. Diffusion limited, will limit the size of such constructs.

Three-dimensional cell-laden hydrogel scaffolds as provided herein include cavities patterned therein. Such cavities perform vascular functions, such as providing nutrients and oxygen to cells and removing waste. As a result, provided scaffolds having cavities, microvasculature or vasculature, would permit greater access to cells and thereby greatly increase the maximum size at which cell growth could be supported. To investigate the ability of multiphoton micromachining to pattern cell-laden hydrogels, we embedded hMSCs in the bulk of a thin gel. FIG. 8 at panel (E at the insert) shows the word “TUFTS” micromachined into a gel and, less than 4 hours after machining, cells were stained with a live/dead fluorescence assay. Following staining the dishes were examined using confocal microscopy. FIG. 8 at panels (F)-(J) show dead cells in the plane of micromachining with living cells present both directly above and below its machined volume. This was expected as cells are largely transparent to 810 nm light so they should be unaffected by the beam far from the focus. The high temperatures at the focus of the beam are likely responsible for the dead cells found in the micromachined regions.

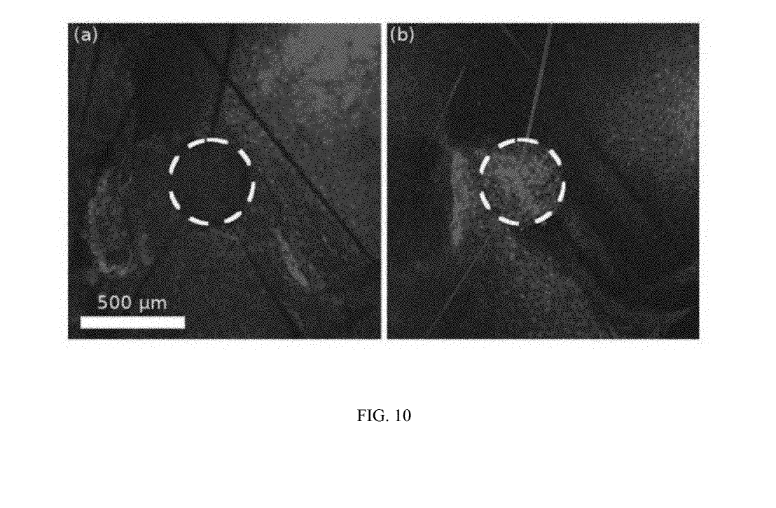

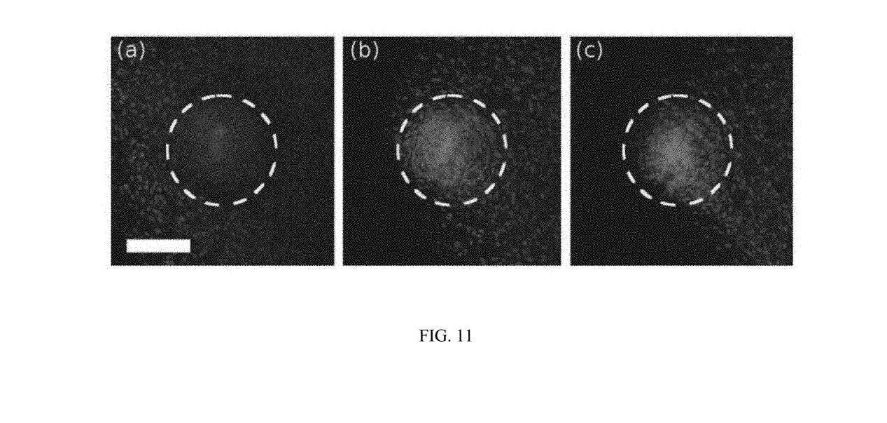

Finally, we conducted a pilot in vivo study in which three mice were implanted with two machined gels each. One gel contained a branching pattern with a main branch diameter of 200 μm; the second gel contained a branching pattern with a main branch diameter of 400 μm. One mouse was killed at each of week 2, week 3, and week 4. Upon subsequent imaging we were able to identify the machined features in four of the six samples with at least one feature identified at each timepoint. FIG. 9, FIG. 10, and FIG. 11 show cells that penetrated the gels via the machined features in the 2 week and 3 week samples. In the 4 week sample, cells were found to have overgrown the machined feature and not penetrate into the gel. It is likely that the overgrowth in the 4 week case was not due to the extra time of implantation as no cells were seen to penetrate the gel, but rather occurred relatively soon after implantation.

These results are significant as they show that multiphoton micromachining in silk fibroin hydrogels was capable of directing cell growth and infiltration into an artificial construct. Patterned biocompatible constructs are of great interest in the field of tissue engineering, which seeks to artificially recapitulate natural structures in the body. One promising avenue to do so is the use of decellularization as a means to replace damaged organs. (See for example, Badylak S F., et al., “Whole-Organ Tissue Engineering: Decellularization and Recellularization of Three-Dimensional Matrix Scaffolds”, 13 Annu Rev Biomed Eng, 27-53 (2011)). This technique involves the harvest of a healthy organ and the removal of all cellular material, leaving behind a structured extracellular matrix. The resulting decellularized scaffold acts as a template for new cell growth. However, this technique requires access to a healthy organ as well as time for cell culture. Whereas this method could be used to reduce rejection of donated organs, it does little to help those who are still waiting for an organ transplant. Whereas the micropatterning described here is too small-scale to be used to replicate an entire organ, it provides a unique combination of high-resolution (micrometer-scale) structuring with the possibility of generating large (nearly millimeter-scale) features. We believe this combination of high resolution with large volume of modification could prove useful to link large-scale three-dimensional patterning of biological materials using techniques like three-dimensional bioprinting (see for example, Murphy S V. and Atala A. “3D Bioprinting of Tissues and Organs”, 32 Nat Biotechnol 8, 773-785 (2014)), with techniques to produce random voids in a material on the 0.1 m scale. (See for example, Bellan L M, et al., “Fabrication of an Artificial 3-Dimensional Vascular Network using Sacrificial Sugar Structures”, 5 Soft Matter, 1354-1357 (2009)).

### Scattering Loss Measurements

Silk solution was filtered through a 0.22 μm pore size filter to remove dust and other scatterers present in the solution. This solution was formed into a gel in a plastic semimicro fluorescence cuvette with 5 mm and 10 mm path lengths. A green laser was propagated through both the long and short paths and the transmitted intensity measured. From these measurements we were able to calculate scattering losses in the gel to be 0.6±0.3 dB/cm. This translates to a 1/e scattering length of between 5 and 14 cm.

### Spot Size Measurement

The spot size at the focus of the microscope objective was measured via the knife edge technique. The beam was focused in the plane of a razor blade which was mounted on a micropositioning stage. A second microscope objective was used to collect light transmitted past the razor blade. During the measurement the razor blade was moved through the focus of the beam and the transmitted intensity measured using a photodiode. The derivative of the measured intensity was fit to a Gaussian function to calculate the spot size of the beam. This measurement was repeated in both the X and Y directions. FIG. 3 shows its beam profile was found to be a good fit for the Gaussian function with a spot size of 5 μm in the X direction (see FIG. 3 at the Top panel) and 6 μm in the Y direction (see FIG. 3 at the Bottom panel). This spot size is larger than what would be expected from a diffraction-limited system because the beam did not fill the rear aperture of the objective.

### Self-Focusing

The symmetrical shape of the features along the optical axis indicates that self-focusing effects are not present using low energy pulses. To obtain an estimate of the critical power for self-focusing (P_{cr}), we used an amplified laser system to examine the shape of the features micromachined by a single pulse using the same 10×, 0.3 NA objective used for oscillator only micromachining. FIG. 4 shows asymmetrical features consistent with self-focusing were observed at pulse energies above 1 μJ. Assuming a 150 fs pulse, this translates to P_{cr} on the order of 6 MW. The peak power of the pulse during micromachining is no more than 60 kW, which is far below the threshold value and explains why self-focusing is avoided.

### In Vivo Implantation

Gels with branching features machined into them were implanted in mice for up to 4 wk. Two sizes of branching features were formed, one with a main branch diameter of 200 μm and the other with a main branch diameter of 400 μm. FIG. 5, FIG. 7, and FIG. 9 show confocal images of branching.

### Other Embodiments and Equivalents

While the present disclosure has explicitly discussed certain particular embodiments and examples of the present disclosure, those skilled in the art will appreciate that the invention is not intended to be limited to such embodiments or examples. On the contrary, the present disclosure encompasses various alternatives, modifications, and equivalents of such particular embodiments and/or example, as will be appreciated by those of skill in the art.

Accordingly, for example, methods and diagrams of should not be read as limited to a particular described order or arrangement of steps or elements unless explicitly stated or clearly required from context (e.g., otherwise inoperable). Furthermore, different features of particular elements that may be exemplified in different embodiments may be combined with one another in some embodiments.

1. A method of forming a biomechanical scaffold, the method comprising steps of:
providing a protein hydrogel;
irradiating the hydrogel with a pulsed beam,
wherein the irradiating step comprises focusing the beam on a spot beneath a surface of the hydrogel;
initiating multiphoton absorption by the hydrogel at the spot;
disrupting the hydrogel at the spot; and
removing at least a portion the hydrogel from the spot to form at least one cavity beneath the surface of the hydrogel.

2. The method of claim 1, wherein the protein hydrogel comprises or consists of silk fibroin.

3. The method of claim 1, wherein the spot is at a depth of at least 200 μm beneath the surface of the hydrogel.

4. (canceled)

5. The method of claim 1, further comprising depositing removed hydrogel on a surface of the at least one cavity.

6. (canceled)

7. The method of claim 1, further comprising shifting a position of the focused beam to extend the at least one cavity from an initial focal spot so that the hydrogel is characterized by a cavity having a shape or pattern beneath the surface of the hydrogel.

8. The method of claim 7, further comprising a step of seeding the cavity with viable cells, wherein the seeded cells penetrate the hydrogel.

9. (canceled)

10. The method of claim 1, wherein the providing step, the protein hydrogel comprises viable cells encapsulated therein.

11. The method of claim 10, wherein when irradiating, the viable cells and the protein hydrogel in an area surrounding the focal spot are transparent to the beam, such that they are not disrupted or removed.

12. (canceled)

13. The method of claim 10, before the providing step, a step of encapsulating cells within the protein hydrogel.

14. The method of claim 7, wherein the step of shifting occurs at a rate of about 0.05 mm/sec to about 10 mm/sec.

15. The method of claim 14, wherein the step of shifting is in a lateral, longitudinal, or normal direction relative to a surface of the hydrogel.

16. A biomechanical scaffold, comprising:
a protein hydrogel;
at least one cavity formed beneath a surface of the hydrogel, wherein the at least one cavity is defined by an interior wall; and
disrupted protein hydrogel deposited on a surface of the interior wall, wherein the deposited hydrogel is characterized in that it was removed from the protein hydrogel when forming the at least one cavity.

17. The biomechanical scaffold of claim 16, wherein the protein hydrogel comprises or consists of silk fibroin.

18. The biomechanical scaffold of claim 16, wherein the protein hydrogel comprises at least 90% water.

19. The biomechanical scaffold of claim 16, wherein the at least one cavity is at least about 200 μm beneath the surface of the hydrogel.

20. (canceled)

21. (canceled)

22. The biomechanical scaffold of claim 16, wherein the at least one cavity extends away from an initial focal spot so that the hydrogel is characterized by a cavity having a shape or pattern beneath the surface of the hydrogel.

23. The biomechanical scaffold of claim 22, wherein the protein hydrogel comprises viable cells and wherein the shape or pattern of the at least one cavity directs cellular growth and is engineered to permit introduction of nutrients and oxygen and removal of waste.

24. (canceled)

25. The biomechanical scaffold of claim 23, characterized in that when the shape or pattern is seeded with the viable cells on the redeposited material, the cells penetrate the hydrogel and spread according to the shape or pattern.

26. (canceled)

27. The biomechanical scaffold of claim 16, further comprising at least one additive, agent, and/or functional moiety.

28. (canceled)

29. A method of directing cell growth in vivo, the method comprising steps of:
providing the biomechanical scaffold of any of the preceding claims;
seeding cells on the deposited hydrogel surface;
allowing the cells to penetrate the hydrogel;
implanting the scaffold in a subject.
